(19) United States
(12) Patent Application Publication
(10) Pub. No.: US 2023/0332247 A1
(43) Pub. Date: Oct. 19, 2023

(54) METHODS, COMPOSITIONS, AND SYSTEMS FOR CAPTURING ANALYTES FROM GLIOBLASTOMA SAMPLES

(71) Applicant: 10x Genomics, Inc., Pleasanton, CA (US)

(72) Inventors: Hardeep Pal Singh, Livermore, CA (US); David Sukovich, Oakland, CA (US); Seayar Mohabbat, Manteca, CA (US); Dongyao Li, Pleasanton, CA (US); Hanyoup Kim, Foster City, CA (US); Augusto Manuel Tentori, Dublin, CA (US)

(21) Appl. No.: 18/340,501

(22) Filed: Jun. 23, 2023

Related U.S. Application Data
(63) Continuation of application No. PCT/US2023/012126, filed on Feb. 1, 2023.
(60) Provisional application No. 63/305,497, filed on Feb. 1, 2022.

(51) Int. Cl. C12Q 1/6886; C12Q 1/6834

(52) U.S. Cl. CPC C12Q 1/6886; C12Q 1/6834; C12Q 2600/158; G01N 2800/7028

(57) ABSTRACT

Provided herein are methods, compositions and systems for identifying spatial gene expression of analytes from glioblastoma derived tissue. The methods discloses herein include using templated ligation probe pairs to identify location of a disease proliferating region in a glioblastoma-derived sample by detecting analytes in the region and hybridizing a ligation product comprising the probe pairs to a capture probe on a spatial array.

### CROSS-REFERENCE TO RELATED APPLICATIONS

Pursuant to 35 U.S.C. § 119(e), this application is a continuation of International Application PCT/US2023/012126, with an international filing date of Feb. 1, 2023, which claims the benefit of U.S. Provisional Patent Application No. 63/305,497, filed Feb. 1, 2022, the contents and disclosures of each application are incorporated herein by reference in their entireties.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue). Spatial analysis of an analyte within a biological sample, particularly in glioblastoma samples, may require determining the sequence of the analyte sequence or a complement thereof and the sequence of the spatial barcode or a complement thereof to identify the location of the analyte. The biological sample may be placed on a solid support to improve specificity and efficiency when being analyzed for identification or characterization of an analyte, such as DNA, RNA or other genetic material, within the sample.

Generally, targeting a particular analyte in a biological sample utilizes a capture probe that targets a common transcript sequence such as a poly(A) mRNA-like tail. However, this approach is capable of detecting a high number of off target analytes. Methods such as RNA-templated ligation offer an alternative to indiscriminant capture of a common transcript sequence. See, e.g., Yeakley, PLoS One, 25; 12(5):e0178302 (2017. However, there remains a need to develop an alternative to common transcript sequence (e.g., poly(A) mRNA-like tail) capture of target analytes that is capable of detecting an analyte(s) in an entire transcriptome while providing information regarding the spatial location and abundance of a target analyte.

Glioblastoma is a common type of malignant brain tumor with a median survival time of 12-14 months. Aside from standard histological assessment of these tumors, RNA sequencing from these diseased tissues can provide insights in gene expression from biomarkers, which could dictate the clinical outcome of a subject. However, standard RNA sequencing workflows require dissociation of the tissue, resulting in the loss of spatial patterns of gene expression. There remains a need to develop methods to identify the spatial location of analytes in glioblastoma samples, which can then be used as a diagnostic tool for, for example, developing personal medicine treatment regimens for a subject.

### SUMMARY

The present disclosure features methods, compositions, devices, and systems for determining the location of an analyte in glioblastoma tissue samples. Determining the spatial location of analytes (e.g., proteins, DNA, or RNA) within a glioblastoma tissue leads to better understanding of spatial heterogeneity in various contexts, such as disease models. In some instances, the techniques disclosed herein facilitate downstream processing, such as sequencing. The methods described herein allow for identification of the grade and/or metastatic status of the glioblastoma sample using expression detection of mRNA analytes.

In some examples, the methods, compositions, devices, and systems disclosed herein utilize RNA-templated ligation (RTL) for analyzing an analyte (e.g., RNA) in a glioblastoma tissue. In some examples, RTL is used in combination with a “sandwich process,” wherein the analyte is transferred from a first substrate to a second substrate for further downstream processing. In some examples, the methods disclosed herein allow spatial analysis of two different types of analytes. The methods in some instances utilize additional means of detection such as immunohistochemistry or immunofluorescence.

Thus, disclosed herein are methods of determining location of an analyte in a glioblastoma sample, the methods comprising: (a) providing the glioblastoma sample on a first substrate; (b) contacting a first probe and a second probe with the glioblastoma sample, wherein the first probe and the second probe each comprise one or more sequences that are substantially complementary to sequences of the analyte, and wherein the second probe comprises a capture probe capture domain; (c) hybridizing the first probe and the second probe to the analyte; (d) generating a ligation product by ligating the first probe and the second probe; (e) releasing the ligated product from the analyte; (f) hybridizing the ligation product to a capture domain of a capture probe on an array; and (g) determining (i) all or a part of the sequence of the ligation product bound to the capture domain, or a complement thereof, and (ii) all of a spatial barcode, or a complement thereof, and using the determined sequence of (i) and (ii) to identify the location of the analyte in the glioblastoma sample.

In some instances, the glioblastoma sample is an archived fixed glioblastoma sample. In some instances, the archived fixed glioblastoma sample has been stored on the first substrate for at least six months. In some instances, the archived fixed glioblastoma sample has been stored on the first substrate for at least one year, at least two years, at least three years, or more.

In some instances, the analyte is selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof. In some instances, the analyte indicates a grade of the glioblastoma tissue. In some instances, the grade is a precursor of glioblastoma, Grade I glioblastoma, Grade II glioblastoma, Grade III glioblastoma, or Grade IV glioblastoma.

In some instances, the archived fixed glioblastoma sample has been stored on the first substrate at room temperature. In some instances, the archived fixed glioblastoma sample has been stored on the first substrate at a temperature above room temperature. In some instances, the biological sample is a formalin-fixed paraffin-embedded (FFPE) glioblastoma sample, a PFA fixed glioblastoma sample, or an acetone fixed glioblastoma sample. In some instances, the glioblastoma sample is an FFPE sample.

In some instances, the first substrate comprises an array comprising a plurality of capture probes, wherein a capture probe of the plurality of capture probes comprises (i) the spatial barcode and (ii) the capture domain. In some instances, the method further comprises: aligning the first substrate with a second substrate comprising an array, such that at least a portion of the glioblastoma sample is aligned with at least a portion of the array, wherein the array comprises a plurality of capture probes, wherein a capture probe of the plurality of capture probes comprises (i) the spatial barcode and (ii) the capture domain; and when the glioblastoma sample is aligned with at least a portion of the array, performing the releasing step (e) and migrating the ligation product from the glioblastoma sample to the array.

In some instances, the first probe and the second probe comprise a contiguous nucleic acid sequence. In some instances, the first probe and the second probe hybridize to adjacent sequences of the analyte. In some instances, the adjacent sequences are at least 1, 2, 3, 4, 5, 6, 7, 8, 9, 10, about 15, about 20, or more nucleotides away from one another.

In some instances, the method further comprises generating an extended first probe, wherein the extended first probe comprises a sequence complementary to a sequence between the sequence hybridized to the first probe and the sequence hybridized to the second probe. In some instances, the method further comprises generating an extended second probe using a polymerase, wherein the extended second probe comprises a sequence complementary to a sequence between the sequence hybridized to the first probe and the sequence hybridized to the second probe.

In some instances, the method further comprises hybridizing a third probe to the first probe and the second probe. In some instances, the third probe comprises: (i) a sequence that is at least 80%, at least 85%, at least 90%, at least 95%, at least 96%, at least 97%, at least 98%, or at least 99%, or 100% complementary to a portion of the first probe that hybridizes to the third probe; and (ii) a sequence that is at least 80%, at least 85%, at least 90%, at least 95%, at least 96%, at least 97%, at least 98%, or at least 99%, or 100% complementary to a portion of the second probe that hybridizes to the third probe.

In some instances, the first probe and the second probe are ligated together to generate a ligation product. In some instances, the ligation product is generated via enzymatic ligation and is performed by an enzyme that is selected from a Chlorella virus DNA ligase, a single stranded DNA ligase, or a T4 DNA ligase.

In some instances, the releasing step (e) comprises contacting the glioblastoma sample with a reagent medium comprising a permeabilization agent and an agent for releasing the ligation product from the target nucleic acid, thereby permeabilizing the biological sample and releasing the ligation product from the analyte. In some instances, the agent for releasing the ligation product from the target nucleic acid comprises a nuclease. In some instances, the nuclease comprises an RNase, optionally wherein the RNase is selected from RNase A, RNase C, RNase H, or RNase I. In some instances, the method further comprises amplifying the ligation product after the releasing step. In some instances, the permeabilization agent comprises a protease. In some instances, the protease is selected from trypsin, pepsin, elastase, or proteinase K. In some instances, the glioblastoma sample and the array are contacted with the reagent medium for about 1 to about 60 minutes.

In some instances, the determining comprises sequencing (i) all or a part of the sequence of the ligation product, or a complement thereof, and (ii) all of the spatial barcode, or a complement thereof. In some instances, the capture probe comprises a poly(T) sequence. In some instances, the capture probe comprises a sequence complementary to the first probe or the second probe. In some instances, the capture probe further comprises one or more functional domains, a unique molecular identifier (UMI), a cleavage domain, and combinations thereof.

In some instances, the analyte is mRNA. In some instances, the mRNA is one or more biomarkers selected from Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.

In some instances, the method further comprises imaging the glioblastoma sample. In some instances, the glioblastoma sample is imaged using immunofluorescence.

Also provided herein are methods of determining the location of a disease proliferating region in a glioblastoma derived sample, comprising: (a) providing the glioblastoma derived sample on a first substrate; (b) contacting a first probe and a second probe with the glioblastoma derived sample, wherein the first probe and the second probe each comprise one or more sequences that are substantially complementary to sequences of an analyte indicative of a disease proliferating region in a glioblastoma, and wherein the second probe comprises a capture probe capture domain; (c) hybridizing the first probe and the second probe to the analyte; (d) generating a ligation product by ligating the first probe and the second probe; (e) releasing the ligated product from the analyte; (f) hybridizing the ligation product to a capture domain; and (g) determining (i) all or a part of the sequence of the ligation product bound to the capture domain, or a complement thereof, and (ii) all of a spatial barcode, or a complement thereof, and using the determined sequence of (i) and (ii) to identify the location of a disease proliferating region in the glioblastoma derived sample.

In some instances, the analyte indicative of a disease proliferating region in a glioblastoma is an mRNA that codes for one or more of Ki67, CCNB1, and MYCC. In some instances, the disease proliferating region is a region of metastasis.

In some instances, the glioblastoma derived sample is a formalin-fixed paraffin-embedded (FFPE) biological sample, a PFA fixed biological sample, or an acetone fixed biological sample. In some instances, the glioblastoma derived sample is an FFPE sample.

In some instances, the first substrate comprises an array comprising a plurality of capture probes, wherein a capture probe of the plurality of capture probes comprises (i) the spatial barcode and (ii) the capture domain. In some instances, the method further comprises: aligning the first substrate with a second substrate comprising an array, such that at least a portion of the glioblastoma derived sample is aligned with at least a portion of the array, wherein the array comprises a plurality of capture probes, wherein a capture probe of the plurality of capture probes comprises (i) the spatial barcode and (ii) the capture domain; and when the glioblastoma derived biological sample is aligned with at least a portion of the array, performing the releasing step (e) and migrating the ligation product from the glioblastoma derived biological sample to the array. In some instances, the first probe and the second probe are on a contiguous nucleic acid sequence. In some instances, the first probe and the second probe hybridize to adjacent sequences of the analyte. In some instances, the adjacent sequences are at least 1, 2, 3, 4, 5, 6, 7, 8, 9, 10, or more nucleotides away from one another.

In some instances, the method further comprises generating an extended first probe, wherein the extended first probe comprises a sequence complementary to a sequence between the sequence hybridized to the first probe and the sequence hybridized to the second probe. In some instances, the method further comprises generating an extended second probe using a polymerase, wherein the extended second probe comprises a sequence complementary to a sequence between the sequence hybridized to the first probe and the sequence hybridized to the second probe.

In some instances, the method further comprises hybridizing a third probe to the first probe and the second probe. In some instances, the third probe comprises: (i) a sequence that is at least 80%, at least 85%, at least 90%, at least 95%, at least 96%, at least 97%, at least 98%, or at least 99%, or 100% complementary to a portion of the first probe that hybridizes to the third probe; and (ii) a sequence that is at least 80%, at least 85%, at least 90%, at least 95%, at least 96%, at least 97%, at least 98%, or at least 99%, or 100% complementary to a portion of the second probe that hybridizes to the third probe.

In some instances, the first probe and the second probe are ligated together to generate a ligation product. In some instances, the ligation product is generated via enzymatic ligation and is performed by an enzyme that is selected from a Chlorella virus DNA ligase, a single stranded DNA ligase, or a T4 DNA ligase.

In some instances, the releasing step (e) comprises contacting the glioblastoma derived sample with a reagent medium comprising a permeabilization agent and an agent for releasing the ligation product from the target nucleic acid, thereby permeabilizing the glioblastoma derived sample and releasing the ligation product from the analyte. In some instances, the agent for releasing the ligation product from the target nucleic acid comprises a nuclease. In some instances, the nuclease comprises an RNase, optionally wherein the RNase is selected from RNase A, RNase C, RNase H, or RNase I.

In some instances, the method further comprises amplifying the ligation product after the releasing step. In some instances, the permeabilization agent comprises a protease. In some instances, the protease is selected from trypsin, pepsin, elastase, or proteinase K. In some instances, the glioblastoma derived sample and the array are contacted with the reagent medium for about 1 to about 60 minutes.

In some instances, the determining comprises sequencing (i) all or a part of the sequence of the ligation product, or a complement thereof, and (ii) all of the spatial barcode, or a complement thereof. In some instances, the capture probe comprises a poly(T) sequence. In some instances, the capture probe comprises a sequence complementary to the first probe or the second probe. In some instances, the capture probe further comprises one or more functional domains, a unique molecular identifier (UMI), a cleavage domain, and combinations thereof.

In some instances, the analyte is mRNA. In some instances, the method further comprises imaging the glioblastoma derived sample. In some instances, the glioblastoma derived sample is imaged using immunofluorescence.

In some instances, the method further comprises processing a second type of analyte in the glioblastoma derived sample. In some instances, the processing the second type of analyte comprises: contacting the glioblastoma derived sample with a plurality of analyte capture agents, wherein an analyte capture agent of the plurality of analyte capture agents comprises an analyte binding moiety and an analyte binding moiety barcode domain, wherein the analyte binding moiety specifically binds to the second type of analyte, and wherein the analyte binding moiety barcode domain comprises an analyte binding moiety barcode and a capture handle sequence; and hybridizing the capture handle sequence to the capture domain of the capture probe on the array.

In some instances, the method further comprises determining (i) all or part of the sequence of the analyte capture moiety barcode domain; and (ii) the spatial barcode, or a complement thereof, and using the determined sequence of (i) and (ii) to analyze the location of the second type of analyte in the glioblastoma derived sample.

In some instances, the method further comprises a releasing step, wherein the analyte moiety barcode domain is released from the analyte binding moiety.

In some instances, the second type of analyte is a protein analyte. In some instances, the protein analyte is one or more protein biomarkers selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.

Also provided herein are compositions comprising: (a) an array comprising a plurality of capture probes affixed to a substrate, wherein a capture probe of the plurality of capture probes comprises (i) a spatial barcode and (ii) a capture domain, wherein the spatial barcode comprises a sequence that provides a location of an analyte in a biological sample; (b) the biological sample placed on the array, wherein the biological sample comprises the analyte; and (c) a ligation product comprising a first probe and a second probe, wherein the first probe and the second probe each comprise a sequence that is substantially complementary to a sequence of the analyte, and wherein one of the first probe or the second probe comprises a capture probe capture domain that is hybridized to the capture domain of the capture probe on the array, wherein the analyte is one or more biomarkers selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof, or any combination thereof.

Also provided herein are compositions comprising: (a) a biological sample placed on a first substrate, wherein the biological sample comprises an analyte; and (b) a second substrate comprising an array comprising a plurality of capture probes affixed to the substrate, wherein a capture probe of the plurality of capture probes comprises (i) a spatial barcode comprising a sequence that provides a location of the analyte and (ii) a capture domain, wherein the first substrate is aligned with the second substrate, such that at least a portion of the biological sample is aligned with at least a portion of the array; (c) a ligation product comprising a first probe and a second probe, wherein the first probe and the second probe each comprise a sequence that is substantially complementary to a sequence of the analyte, and wherein one of the first probe or the second probe comprises a capture probe capture domain that is hybridized to the capture domain of the capture probe on the second substrate, wherein the analyte is one or more biomarkers selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof, or any combination thereof.

In some instances, the composition further comprises 100 or more ligation product pairs, wherein a ligation product pair of the 100 or more ligation product pairs comprises the first probe and the second probe that are ligated together, and wherein at least one of the ligation product pairs is hybridized to the capture domain of the capture probe.

Also provided herein are systems for analyzing an analyte in a biological sample, the system comprising: (a) a support device configured to retain a first substrate and a second substrate, wherein the biological sample is placed on the first substrate, and wherein the second substrate comprises a plurality of capture probes, wherein a capture probe of the plurality of capture probes comprises: (i) a spatial barcode and (ii) a capture domain; (b) (b1) a first probe and a second probe, wherein the first probe and the second probe each comprise a sequence that is substantially complementary to adjacent sequences of the analyte, wherein the second probe comprises a capture probe binding domain, and wherein the first probe and the second probe are capable of being ligated together to form a ligation product; or (b2) a plurality of analyte capture agents, wherein an analyte capture agent of the plurality of analyte capture agents comprises an analyte binding moiety and an analyte binding moiety barcode domain, wherein the analyte binding moiety specifically binds to the analyte, and wherein the analyte binding moiety barcode domain comprises an analyte binding moiety barcode and a capture handle sequence; (c) a reagent medium comprising a permeabilization agent and optionally an agent for releasing the ligation product; and (d) instructions for performing any one of the methods described herein, wherein the analyte is selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.

Also disclosed herein is a method of determining location, or spatial location of an analyte in a biological sample, wherein the biological sample is a glioblastoma sample, the method comprising: (a) providing the biological sample on a first substrate; (b) contacting a first probe and a second probe with the biological sample, wherein the first probe and the second probe each comprise one or more sequences that are substantially complementary to sequences of the analyte, and wherein the second probe comprises a capture probe capture domain; (c) hybridizing the first probe and the second probe to the analyte; (d) generating a ligation product by ligating the first probe and the second probe; (e) releasing the ligated product from the analyte; (f) hybridizing the ligation product to a capture domain; and (g) determining (i) all or a part of the sequence of the ligation product bound to the capture domain, or a complement thereof, and (ii) all of a spatial barcode, or a complement thereof, and using the determined sequence of (i) and (ii) to identify the location of the analyte in the biological sample.

In some instances, the biological sample is an archived fixed biological sample. In some instances, the archived fixed biological sample has been stored on the first substrate for at least six months. In some instances, the archived fixed biological sample has been stored on the first substrate for at least one year, at least two years, at least three years, or more.

In some instances, the analyte is selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.

In some instances, the analyte indicates a grade of the glioblastoma tissue. In some instances, the grade is a precursor of glioblastoma, Grade I glioblastoma, Grade II glioblastoma, Grade III glioblastoma, or Grade IV glioblastoma.

In some instances, the archived fixed biological sample has been stored on the first substrate at room temperature. In some instances, the archived fixed biological sample has been stored on the first substrate at a temperature above room temperature. In some instances, the biological sample is a formalin-fixed paraffin-embedded (FFPE) biological sample, a PFA fixed biological sample, or an acetone fixed biological sample. In some instances, the biological sample is an FFPE sample.

In some instances, the first substrate comprises an array comprising a plurality of capture probes, wherein a capture probe of the plurality of capture probes comprises (i) the spatial barcode and (ii) the capture domain.

In some instances, the methods further includes aligning the first substrate with a second substrate comprising an array, such that at least a portion of the biological sample is aligned with at least a portion of the array, wherein the array comprises a plurality of capture probes, wherein a capture probe of the plurality of capture probes comprises (i) the spatial barcode and (ii) the capture domain; and when the biological sample is aligned with at least a portion of the array, performing the releasing step (e) and migrating the ligation product from the biological sample to the array.

In some instances, the first probe and the second probe are on a contiguous nucleic acid sequence. In some instances, the first probe and the second probe hybridize to adjacent sequences of the analyte. In some instances, the adjacent sequences are at least 1, 2, 3, 4, 5, 6, 7, 8, 9, 10, or more nucleotides away from one another.

In some instances, the methods further include an extended first probe, wherein the extended first probe comprises a sequence complementary to a sequence between the sequence hybridized to the first probe and the sequence hybridized to the second probe. In some instances, the methods further include generating an extended second probe using a polymerase, wherein the extended second probe comprises a sequence complementary to a sequence between the sequence hybridized to the first probe and the sequence hybridized to the second probe. In some instances, the methods further include hybridizing a third probe to the first probe and the second probe.

In some instances, the third probe comprises: (i) a sequence that is at least 80%, at least 85%, at least 90%, at least 95%, at least 96%, at least 97%, at least 98%, or at least 99%, or 100% complementary to a portion of the first probe that hybridizes to the third probe; and (ii) a sequence that is at least 80%, at least 85%, at least 90%, at least 95%, at least 96%, at least 97%, at least 98%, or at least 99%, or 100% complementary to a portion of the second probe that hybridizes to the third probe.

In some instances, the first probe and the second probe are ligated together to generate a ligation product. In some instances, the ligation product is generated via enzymatic ligation and is performed by an enzyme that is selected from a Chlorella virus DNA ligase, a single stranded DNA ligase, or a T4 DNA ligase. In some instances, the methods further include amplifying the ligation product after the releasing step.

In some instances, the releasing step (e) comprises contacting the biological sample with a reagent medium comprising a permeabilization agent and an agent for releasing the ligation product from the target nucleic acid, thereby permeabilizing the biological sample and releasing the ligation product from the analyte.

In some instances, the agent for releasing the ligation product from the target nucleic acid comprises a nuclease. In some instances, the nuclease comprises an RNase, optionally wherein the RNase is selected from RNase A, RNase C, RNase H, or RNase I. In some instances, the permeabilization agent comprises a protease. In some instances, the protease is selected from trypsin, pepsin, elastase, or proteinase K. In some instances, the biological sample and the array are contacted with the reagent medium for about 1 to about 60 minutes.

In some instances, the determining comprises sequencing (i) all or a part of the sequence of the ligation product, or a complement thereof, and (ii) all of the spatial barcode, or a complement thereof.

In some instances, the capture probe comprises a poly(T) sequence. In some instances, the capture probe comprises a sequence complementary to the first probe or the second probe. In some instances, the capture probe further comprises one or more functional domains, a unique molecular identifier (UMI), a cleavage domain, and combinations thereof.

In some instances, the analyte is mRNA. In some instances, the mRNA is one or more biomarkers selected from Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.

In some instances, the methods further include imaging the biological sample. In some instances, the biological sample is imaged using immunofluorescence.

Also disclosed herein is a method of determining the location of a disease proliferating region in a glioblastoma derived biological sample, comprising: (a) providing the glioblastoma derived biological sample on a first substrate; (b) contacting a first probe and a second probe with the biological sample, wherein the first probe and the second probe each comprise one or more sequences that are substantially complementary to sequences of an analyte indicative of a disease proliferating region in a glioblastoma, and wherein the second probe comprises a capture probe capture domain; (c) hybridizing the first probe and the second probe to the analyte; (d) generating a ligation product by ligating the first probe and the second probe; (e) releasing the ligated product from the analyte; (f) hybridizing the ligation product to a capture domain; and (g) determining (i) all or a part of the sequence of the ligation product bound to the capture domain, or a complement thereof, and (ii) all of a spatial barcode, or a complement thereof, and using the determined sequence of (i) and (ii) to identify the location of a disease proliferating region in the glioblastoma derived biological sample.

In some instances, the analyte indicative of a disease proliferating region in a glioblastoma is an mRNA that codes for one or more of Ki67, CCNB1, and MYCC. In some instances, the disease proliferating region is a region of metastasis. In some instances, the glioblastoma derived biological sample is a formalin-fixed paraffin-embedded (FFPE) biological sample, a PFA fixed biological sample, or an acetone fixed biological sample. In some instances, the glioblastoma derived biological sample is an FFPE sample.

In some instances, the first substrate comprises an array comprising a plurality of capture probes, wherein a capture probe of the plurality of capture probes comprises (i) the spatial barcode and (ii) the capture domain.

In some instances, the methods further include aligning the first substrate with a second substrate comprising an array, such that at least a portion of the glioblastoma derived biological sample is aligned with at least a portion of the array, wherein the array comprises a plurality of capture probes, wherein a capture probe of the plurality of capture probes comprises (i) the spatial barcode and (ii) the capture domain; and when the glioblastoma derived biological sample is aligned with at least a portion of the array, performing the releasing step (e) and migrating the ligation product from the glioblastoma derived biological sample to the array.

In some instances, the first probe and the second probe are on a contiguous nucleic acid sequence. In some instances, the first probe and the second probe hybridize to adjacent sequences of the analyte. In some instances, the adjacent sequences are at least 1, 2, 3, 4, 5, 6, 7, 8, 9, 10, or more nucleotides away from one another. In some instances, the methods further include comprising generating an extended first probe, wherein the extended first probe comprises a sequence complementary to a sequence between the sequence hybridized to the first probe and the sequence hybridized to the second probe.

In some instances, the methods further include generating an extended second probe using a polymerase, wherein the extended second probe comprises a sequence complementary to a sequence between the sequence hybridized to the first probe and the sequence hybridized to the second probe. In some instances, the methods further include hybridizing a third probe to the first probe and the second probe.

In some instances, the third probe comprises: (i) a sequence that is at least 80%, at least 85%, at least 90%, at least 95%, at least 96%, at least 97%, at least 98%, or at least 99%, or 100% complementary to a portion of the first probe that hybridizes to the third probe; and (ii) a sequence that is at least 80%, at least 85%, at least 90%, at least 95%, at least 96%, at least 97%, at least 98%, or at least 99%, or 100% complementary to a portion of the second probe that hybridizes to the third probe.

In some instances, the first probe and the second probe are ligated together to generate a ligation product. In some instances, the ligation product is generated via enzymatic ligation and is performed by an enzyme that is selected from a Chlorella virus DNA ligase, a single stranded DNA ligase, or a T4 DNA ligase. In some instances, the releasing step (e) comprises contacting the glioblastoma derived biological sample with a reagent medium comprising a permeabilization agent and an agent for releasing the ligation product from the target nucleic acid, thereby permeabilizing the glioblastoma derived biological sample and releasing the ligation product from the analyte. In some instances, the agent for releasing the ligation product from the target nucleic acid comprises a nuclease.

In some instances, the nuclease comprises an RNase, optionally wherein the RNase is selected from RNase A, RNase C, RNase H, or RNase I. In some instances, the methods further include amplifying the ligation product after the releasing step. In some instances, the permeabilization agent comprises a protease. In some instances, the protease is selected from trypsin, pepsin, elastase, or proteinase K. In some instances, the glioblastoma derived biological sample and the array are contacted with the reagent medium for about 1 to about 60 minutes. In some instances, the determining comprises sequencing (i) all or a part of the sequence of the ligation product, or a complement thereof, and (ii) all of the spatial barcode, or a complement thereof.

In some instances, the capture probe comprises a poly(T) sequence. In some instances, the capture probe comprises a sequence complementary to the first probe or the second probe. In some instances, the capture probe further comprises one or more functional domains, a unique molecular identifier (UMI), a cleavage domain, and combinations thereof.

In some instances, the analyte is mRNA. In some instances, the methods further include imaging the glioblastoma derived biological sample. In some instances, the glioblastoma derived biological sample is imaged using immunofluorescence.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows and exemplary workflow for performing a templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIGS. 12A-12E show gene expression in a human glioblastoma tissue section; FIG. 12A shows TAF11L1 mRNA gene expression is a defined area in the human glioblastoma tissue section; FIG. 12B shows Ki67 mRNA gene expression is a defined area in the human glioblastoma tissue section; FIG. 12C shows CCNB1 mRNA gene expression is a defined area in the human glioblastoma tissue section. FIG. 12D shows MYCC mRNA gene expression is a defined area in the human glioblastoma tissue section. FIG. 12E shows MYCN mRNA gene expression is a defined area in the human glioblastoma tissue section.

FIGS. 13A-13C show gene expression data from an exemplary human glioblastoma tissue section; FIG. 13A shows gene clustering (Clusters 1-5), FIG. 13B shows a t-SNE projection UMAP of the five different clusters seen in FIG. 13A, and FIG. 13C shows a graph of median panel gene count and unique molecular identifier (UMI) count in the human glioblastoma tissue section.

FIGS. 14A-14C show gene expression in a human glioblastoma tissue section; FIG. 14A shows SYT1 mRNA gene expression is a defined area in the human glioblastoma tissue section; FIG. 14B shows SLC12A5 mRNA gene expression is a defined area in the human glioblastoma tissue section; FIG. 14C shows GABRA1 mRNA gene expression is a defined area in the human glioblastoma tissue section.

FIGS. 15A and 15B show gene expression in a human glioblastoma section for EGFR and AKT1, respectively.

FIGS. 16A-16C show gene expression in a human glioblastoma tissue section; FIG. 16A shows RELA mRNA gene expression is a defined area in the human glioblastoma tissue section; FIG. 16B shows NFKB1 mRNA gene expression is a defined area in the human glioblastoma tissue section; FIG. 16C shows MAPK1 mRNA gene expression is a defined area in the human glioblastoma tissue section.

FIGS. 17A-7B show gene expression in a human glioblastoma tissue section; FIG. 17A shows CXCL8 mRNA gene expression is a defined area in the human glioblastoma tissue section; FIG. 17B shows CD163 mRNA gene expression is a defined area in the human glioblastoma tissue section.

FIGS. 18A-18D show gene expression in a human glioblastoma tissue section; FIG. 18A shows PROM1 mRNA gene expression is a defined area in the human glioblastoma tissue section; FIG. 18B shows L1CAM mRNA gene expression is a defined area in the human glioblastoma tissue section; FIG. 18C shows CD44 mRNA gene expression is a defined area in the human glioblastoma tissue section. FIG. 18D shows CD68 mRNA gene expression is a defined area in the human glioblastoma tissue section.

### DETAILED DESCRIPTION

### I. Introduction

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

The biological sample as used herein can be any suitable biological sample described herein or known in the art. In some embodiments, the biological sample is a tissue. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed using cryosectioning. In some embodiments, the methods further comprise a thawing step, after the cryosectioning.

The biological sample can be from a mammal. In some instances, the biological sample is from a human, mouse, or rat. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, or cell lines.

In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning. In some instances, the biological sample is not fixed with paraformaldehyde (PFA). In some instances, when the biological sample is fixed with a fixative including an alcohol (e.g., methanol or acetone-methanol mixture), it is not decrosslinked afterward. In some preferred embodiments, the biological sample is fixed with a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample e.g., using acetone and/or alcohol (e.g., methanol, acetone-methanol) is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing e.g., by formalin or PFA, the biological sample is dehydrated via sucrose gradient. In some instances, the fixed biological sample is treated with a sucrose gradient and then embedded in a matrix e.g., OCT compound. In some instances, the fixed biological sample is not treated with a sucrose gradient, but rather is embedded in a matrix e.g., OCT compound after fixation. In some embodiments when a fixed frozen tissue sample is treated with a sucrose gradient, it can be rehydrated with an ethanol gradient. In some embodiments, the PFA or formalin fixed biological sample, which can be optionally dehydrated via sucrose gradient and/or embedded in OCT compound, is then frozen e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated in an ethanol gradient.

In some instances, the biological sample (e.g., a fixed frozen tissue sample) is treated with a citrate buffer. Citrate buffer can be used for antigen retrieval to decrosslink antigens and fixation medium in the biological sample. Thus, any suitable decrosslinking agent can be used in addition to or alternatively to citrate buffer. In some embodiments, for example, the biological sample (e.g., a fixed frozen tissue sample) is decrosslinked with TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, it is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, it can be rehydrated with an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLoS One; 6(11):e27704 (2011); and Mathieson W. et al., Am J Clin Pathol.; 146(1):25-40 (2016), for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample is fixed, for example in methanol, acetone, acetone-methanol, PFA, PAXgene or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and target sequences intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. Additional methods of visualization and imaging are known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding the primer to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label selected from the group consisting of a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663. Briefly, in any of the methods described herein, the method includes a step of permeabilizing the biological sample. For example, the biological sample can be permeabilized to facilitate transfer of the extension products to the capture probes on the array. In some embodiments, the permeabilizing includes the use of an organic solvent (e.g., acetone, ethanol, and methanol), a detergent (e.g., saponin, Triton X100™, Tween-20™, or sodium dodecyl sulfate (SDS)), an enzyme (an endopeptidase, an exopeptidase, a protease), or combinations thereof. In some embodiments, the permeabilizing includes the use of an endopeptidase, a protease, SDS, polyethylene glycol tert-octylphenyl ether, polysorbate 80, and polysorbate 20, N-lauroylsarcosine sodium salt solution, saponin, Triton X-100™, Tween-20™, or combinations thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663. Generation of capture probes can be achieved by any appropriate method, including those described in Section (II)(d)(ii) of PCT Publication No. WO2020/176788 and/or U.S. Patent Application Publication No. 2020/0277663.

In some instances, a capture probe and an analyte (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other, and can be interrupted by one or more non-complementary residues within the complementary nucleic acid sequence. In some embodiments, at least 60%, but less than 100%, of the residues of one of the two complementary nucleic acid sequences are complementary to residues in the other nucleic acid sequence. In some embodiments, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence. In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

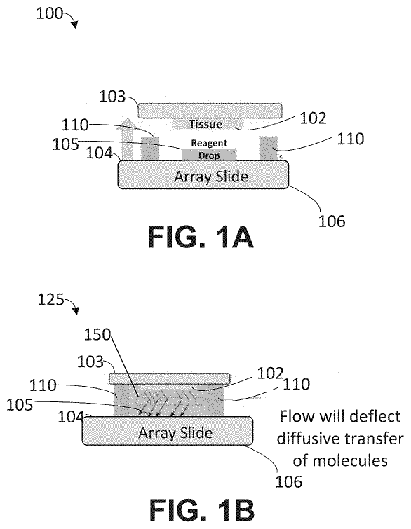

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

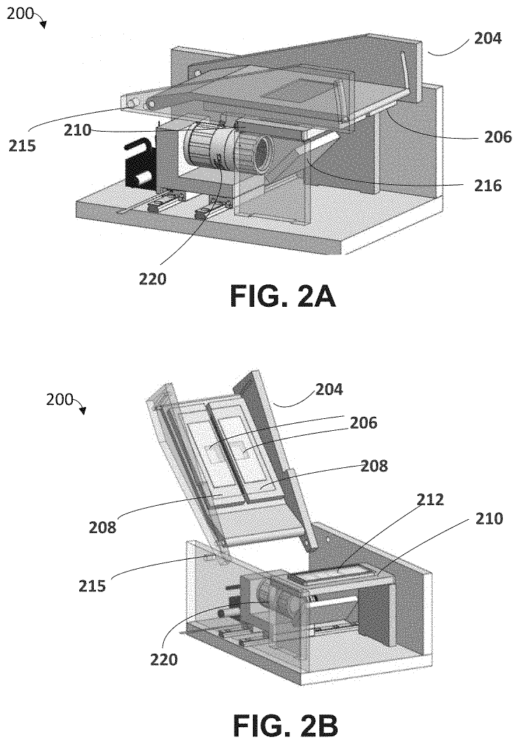

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

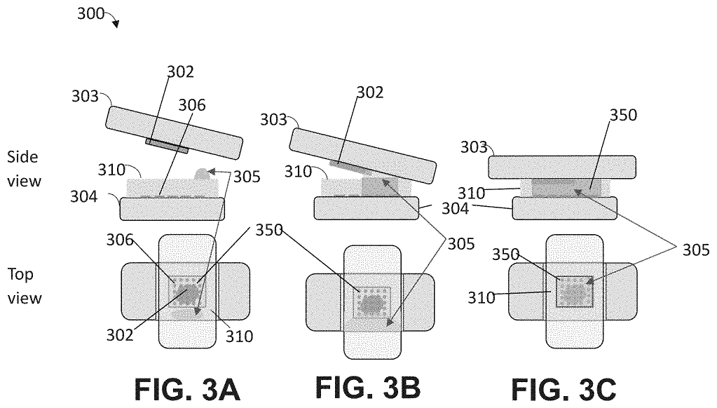

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., the slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

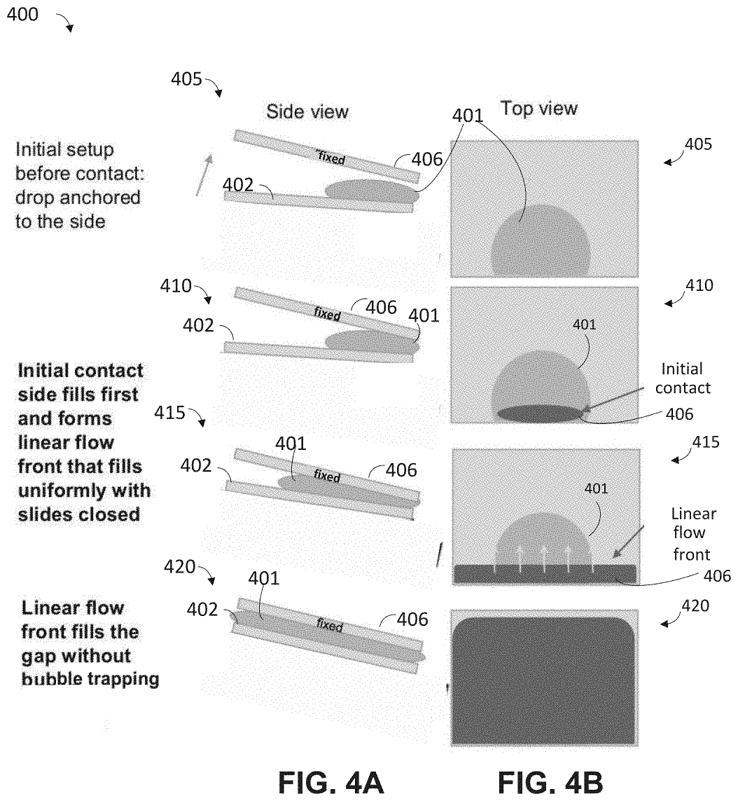

FIG. 4A is a side view of the angled closure workflow 400 in accordance with some exemplary implementations. FIG. 4B is a top view of the angled closure workflow 400 in accordance with some exemplary implementations. As shown at 405, reagent medium 401 is positioned to the side of the substrate 402 contacting the spring.

At step 410, the dropped side of the angled substrate 406 contacts the reagent medium 401 first. The contact of the substrate 406 with the reagent medium 401 may form a linear or low curvature flow front that fills uniformly with the slides closed.

At step 415, the substrate 406 is further lowered toward the substrate 402 (or the substrate 402 is raised up toward the substrate 406) and the dropped side of the substrate 406 may contact and may urge the reagent medium toward the side opposite the dropped side and creating a linear or low curvature flow front that may prevent or reduce bubble trapping between the substrates.

At step 420, the reagent medium 401 fills the gap between the substrate 406 and the substrate 402. The linear flow front of the liquid reagent may form by squeezing the 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, pepsin, elastase, and proteinase K. In some embodiments, the reagent medium comprises a nuclease. In some embodiments, the nuclease comprises an RNase. In some embodiments, the Rnase is selected from Rnase A, Rnase C, Rnase H, and Rnase I. In some embodiments, the reagent medium comprises one or more of sodium dodecyl sulfate (SDS) or a sodium salt thereof, proteinase K, pepsin, N-lauroylsarcosine, and RNAse.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the biological sample and the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for the template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended by a reverse transcriptase. In some embodiments, the capture probe is extended using one or more DNA polymerases. In some embodiments, the extended capture probes include the sequence of the capture domain and the sequence of the spatial barcode of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663. Some quality control measures are described in Section (II)(h) of PCT Publication No. WO2020/176788 and/or U.S. Patent Application Publication No. 2020/0277663.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up- and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663.

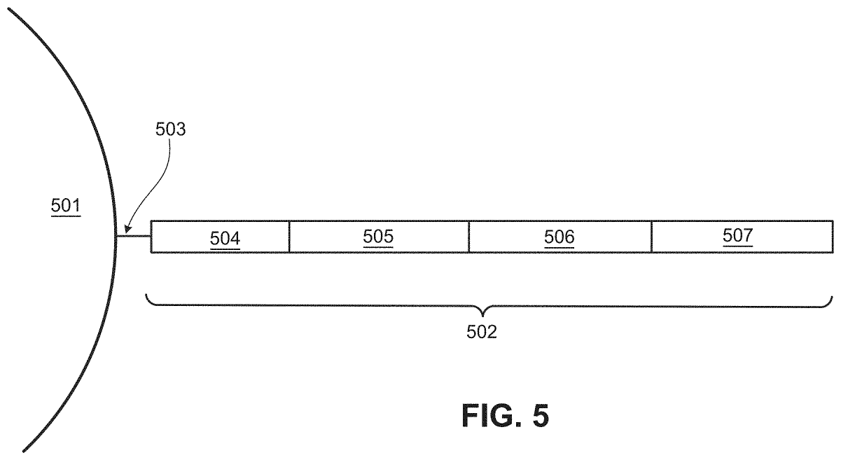

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that are useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

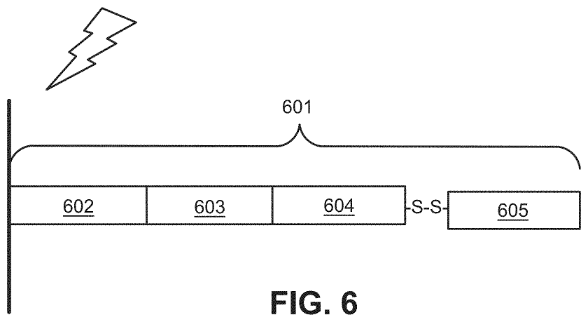

FIG. 6 is a schematic illustrating a cleavable capture probe, wherein the cleaved capture probe can enter into a non-permeabilized cell and bind to analytes within the sample. The capture probe 601 contains a cleavage domain 602, a cell penetrating peptide 603, a reporter molecule 604, and a disulfide bond (—S—S—). 605 represents all other parts of a capture probe, for example a spatial barcode and a capture domain.

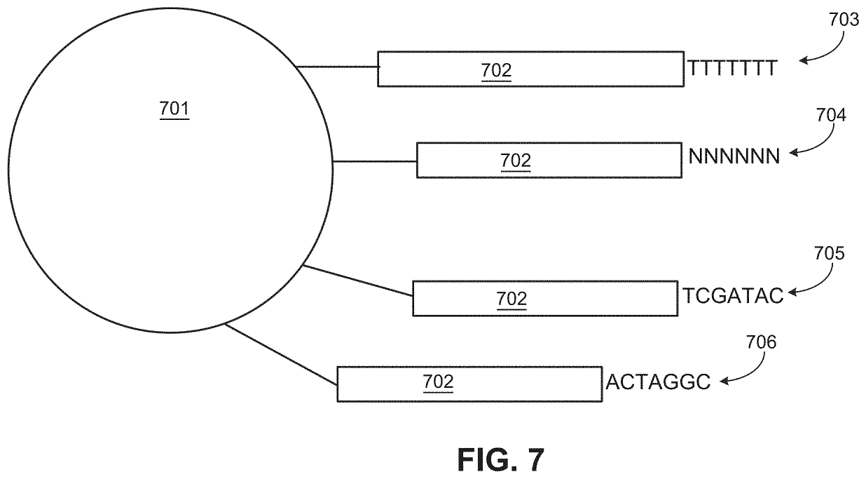

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 702. One type of capture probe associated with the feature includes the spatial barcode 702 in combination with a poly(T) capture domain 703, designed to capture mRNA target analytes. A second type of capture probe associated with the feature includes the spatial barcode 702 in combination with a random N-mer capture domain 704 for gDNA analysis. A third type of capture probe associated with the feature includes the spatial barcode 702 in combination with a capture domain complementary to the analyte capture agent of interest 705. A fourth type of capture probe associated with the feature includes the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequences 504 is common to all of the probes attached to a given feature. In some embodiments, the UMI sequence 506 of a capture probe attached to a given feature is different from the UMI sequence of a different capture probe attached to the given feature.

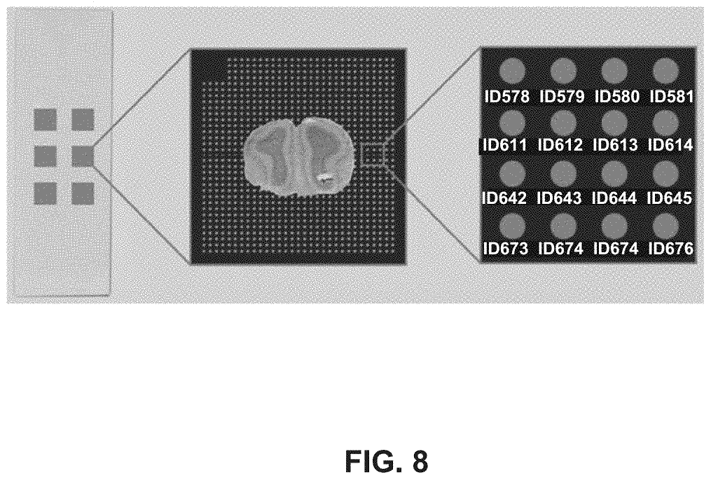

FIG. 8 depicts an exemplary arrangement of barcoded features within an array. From left to right, FIG. 8 shows (L) a slide including six spatially-barcoded arrays, (C) an enlarged schematic of one of the six spatially-barcoded arrays, showing a grid of barcoded features in relation to a biological sample, and (R) an enlarged schematic of one section of an array, showing the specific identification of multiple features within the array (labelled as ID578, ID579, ID560, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture binding capture domain (e.g., a poly(A) sequence, a non-homopolymeric sequence). After hybridization to the analyte, a ligase (e.g., a T4 RNA ligase (Rnl2), a PBCV-1 DNA Ligase or Chorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location and optionally the abundance of the analyte in the biological sample.

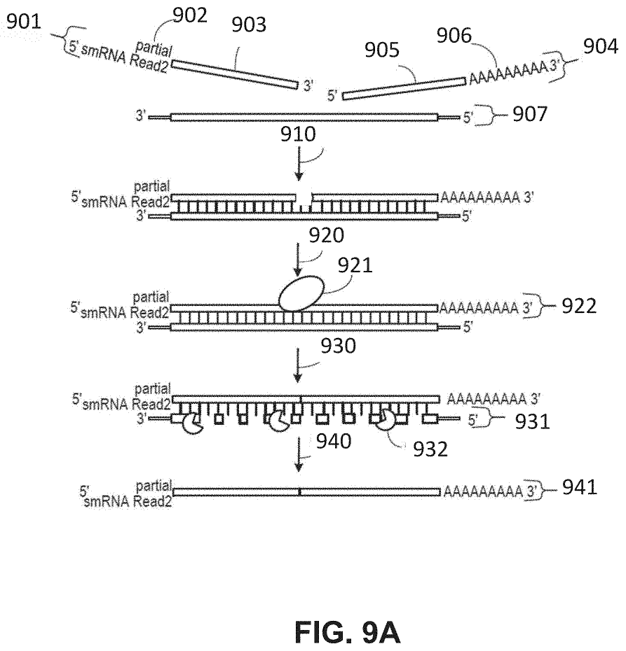

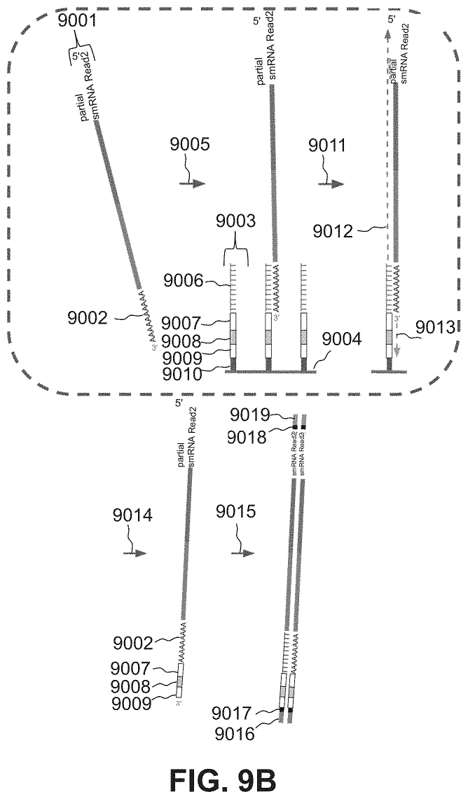

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly-A sequence) 906, the first probe 901 and a second probe 904 hybridize 910 to an analyte 907. A ligase 921 ligates 920 the first probe to the second probe thereby generating a ligation product 922. The ligation product is released 930 from the analyte 931 by digesting the analyte using an endoribonuclease 932. The sample is permeabilized 940 and the ligation product 941 is able to hybridize to a capture probe on the substrate. Methods and composition for spatial detection using templated ligation have been described in PCT Publ. No. WO 2021/133849 A1, U.S. Pat. Nos. 11,332,790 and 11,505,828.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily bind to the captured ligated probe (i.e., compared to no permeabilization). In some embodiments, reverse transcription (RT) reagents can be added to permeabilized biological samples. Incubation with the RT reagents can extend the capture probes 9011 to produce spatially-barcoded full-length cDNA 9012 and 9013 from the captured analytes (e.g., polyadenylated mRNA). Second strand reagents (e.g., second strand primers, enzymes) can be added to the biological sample on the slide to initiate second strand synthesis.

In some embodiments, cDNA can be denatured 9014 from the capture probe template and transferred (e.g., to a clean tube) for amplification, and/or library construction. The spatially-barcoded, full-length cDNA can be amplified 9015 via PCR prior to library construction. The cDNA can then be enzymatically fragmented and size-selected in order to optimize the cDNA amplicon size. P5 9016, i5 9017, i7 9018, and P7 9019, and can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663.

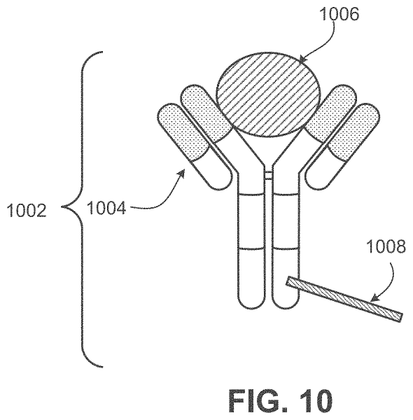

FIG. 10 is a schematic diagram of an exemplary analyte capture agent 1002 comprised of an analyte-binding moiety 1004 and an analyte-binding moiety barcode domain 1008. The exemplary analyte-binding moiety 1004 is a molecule capable of binding to an analyte 1006 and the analyte capture agent is capable of interacting with a spatially-barcoded capture probe. The analyte-binding moiety can bind to the analyte 1006 with high affinity and/or with high specificity. The analyte capture agent can include an analyte-binding moiety barcode domain 1008, a nucleotide sequence (e.g., an oligonucleotide), which can hybridize to at least a portion or an entirety of a capture domain of a capture probe. The analyte-binding moiety barcode domain 1008 can comprise an analyte binding moiety barcode and a capture handle sequence described herein. The analyte-binding moiety 1004 can include a polypeptide and/or an aptamer. The analyte-binding moiety 1004 can include an antibody or antibody fragment (e.g., an antigen-binding fragment).

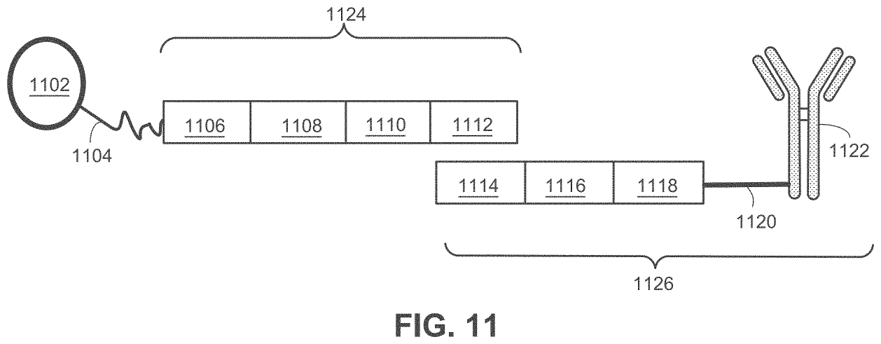

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequences 1106 and UMI 1110, as described elsewhere herein. The capture probe can be affixed 1104 to a feature (e.g., bead) or array 1102. The capture probe can also include a capture domain 1112 that is capable of binding to an analyte capture agent 1126. The analyte capture agent 1126 can include a functional sequence 1118, analyte binding moiety barcode 1116, and a capture handle sequence 1114 that is capable of binding to the capture domain 1112 of the capture probe 1124. The analyte capture agent can also include a linker 1120 that allows the capture agent barcode domain 1116 to couple to the analyte binding moiety 1122.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663. See also, e.g., the Visium Spatial Gene Expression Reagent Kits User Guide (e.g., Rev F, dated January 2022); and/or the Visium Spatial Gene Expression Reagent Kits—Tissue Optimization User Guide (e.g., Rev E, dated February 2022).

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods, Compositions, Devices, and Systems for Capturing Analytes and Derivatives Thereof

### (a) Introduction

Provided herein are methods, devices, compositions, and systems for analyzing the location and abundance of a nucleic acid in a biological sample from a glioblastoma sample. In some instances, the methods include aligning (i.e., sandwiching) a first substrate having the biological sample with a second substrate that includes a plurality of capture probes, thereby “sandwiching” the biological sample between the two substrates. Upon interaction of the biological sample with the substrate having a plurality of probes (in either instance), the location and abundance of a nucleic acid in a biological sample can be determined, as provided herein. This methods includes an advantage in that steps provided herein prior to analyte or analyte-derived molecule by the capture probe, most—if not all—steps can be performed on a substrate that does not have capture probes, thereby providing a method that is cost effective.

The methods disclosed herein further include steps where no sandwiching is performed. Instead, as an exemplary embodiment of the disclosure, the methods include contacting a biological sample with an array of spatially-barcoded capture probes. In some instances, the array is on a substrate and the array includes a plurality of capture probes, wherein a capture probe of the plurality includes: (i) a spatial barcode and (ii) a capture domain. After placing the biological sample on the array, the biological sample is contacted with a first probe and a second probe, wherein the first probe and the second probe each include one or more sequences that are substantially complementary to sequences of the analyte, and wherein the second probe includes a capture probe capture domain; the first probe and the second probe hybridize to complementary sequences in the analyte. After hybridization a ligation product comprising the first probe and the second probe is generated, and the ligation product is released from the analyte. The liberated ligation product, a proxy of the target nucleic acid, is then freed to hybridize to the capture domain of a probe on the array. In some embodiments, each of the preceding steps are performed on a single substrate (i.e., without following the sandwiching process). After capture, (i) all or a part of the sequence of the ligation product specifically hybridized to the capture domain, or a complement thereof, and (ii) all of the spatial barcode, or a complement thereof can be determined, and then one can use the determined sequence of (i) and (ii) to identify the location of the analyte in the biological sample.

The methods and systems provided herein can be applied to an analyte or an analyte-derived molecule(s). As used herein, an analyte derived molecule includes, without limitation, a ligation product from an RNA-templated ligation (RTL) assay, a product of reverse transcription (e.g., an extended capture probe), and an analyte binding moiety barcode (e.g., a binding moiety barcode that identifies that analyte binding moiety (e.g., an antibody)). In some embodiments, the analyte or analyte derived molecules comprise RNA and/or DNA.

In some instances, the methods, devices, compositions, and systems disclosed herein provide efficient release of an analyte or analyte derived molecule from a biological sample so that it can be easily captured or detected using methods disclosed herein.

In some instances, the methods, devices, compositions, and systems disclosed herein allow for detection of analytes or analyte derived molecules from different biological samples using a single array comprising a plurality of capture probes. As such, in some instances, the methods, devices, compositions, and systems allow for serial capture of analytes or analyte derived molecules from multiple samples. The analytes or analyte derived molecules can then be demultiplexed using biological-sample-specific index sequences to identify which serial section expressed the analyte.

In some instances, methods described herein can be used to determine a level and/or at least one activity of one or more biomarkers of glioblastoma. In some instances, the methods can include detecting expression of a first biomarker in a biological sample and then detecting colocalized expression of various second biomarkers with the first biomarker. Provided herein are methods of diagnosing a subject as having glioblastoma. Also provided herein are methods of identifying a subject as having an increased likelihood of having glioblastoma. Further provided herein are methods of monitoring the progression of glioblastoma in a subject. Also provided herein are methods for determining the efficacy of a treatment for glioblastoma in a subject. Further provided herein are methods for treating glioblastoma in a subject. In some embodiments, provided herein are methods of determining efficacy of treatment of a treatment for glioblastoma in a subject. Treatment for glioblastoma to the subject, can include—without limitation—one or more of surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof.

Embodiments of the methods, devices, compositions, and systems disclosed herein are provided below.

### (A) Exemplary Biological Samples from a Glioblastoma Sample

The biological sample as used herein can be any suitable biological sample described herein or known in the art from a glioblastoma sample. In some embodiments, the biological sample is a tissue from a glioblastoma sample. In some embodiments, the tissue sample is a tumor sample from glioblastoma sample. In some embodiments, the biological sample is a tissue section from a glioblastoma sample. In some embodiments, the tissue is from a glioblastoma sample and is flash-frozen and sectioned. Any suitable methods described herein or known in the art can be used to flash-freeze and section the tissue sample. In some embodiments, the biological sample, e.g., the tissue, is flash-frozen using liquid nitrogen before sectioning. In some embodiments, the sectioning is performed using cryosectioning. In some embodiments, the methods further comprises a thawing step, after the cryosectioning. In some embodiments, the biological sample, e.g., the tissue sample is fixed, for example in methanol, acetone, PFA or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RTL methods disclosed herein. A limitation of direct RNA capture for fixed samples is that the RNA integrity of fixed (e.g., FFPE) samples can be lower than a fresh sample, thereby making it more difficult to capture RNA directly, e.g., by capture of a common sequence such as a poly(A) tail of an mRNA molecule. However, by utilizing RTL probes that hybridize to RNA target sequences in the transcriptome, one can avoid a requirement for RNA analytes to have both a poly(A) tail and full length mRNA. Accordingly, RTL probes can be utilized to beneficially improve capture and spatial analysis of fixed samples.

Disclosed herein are methods and compositions for detecting “biomarkers of glioblastoma” includes biomarkers of microglial cells. In some instances, the microglial cells are associated with glioblastoma. Microglia are innate immune cells in the central nervous system that make up a substantial portion of the tumor mass in gliomas, including glioblastomas (Abels, Erik Ret al. (2019) Cell reports vol. 28,12: 3105-3119). Glioblastomas are capable of interacting with microglia, which contributes to the growth of these tumors (Matias D et al., (2017) Reviews on Cancer, 1868(1): 333-340). Methods of detecting biomarkers of glioblastoma have been disclosed previously in US 2021/0222253 A1.

In some instances, the biological sample co-expresses one or more biological markers that can be detected. For instance, one can perform both spatial analysis as described herein (e.g., using templated ligation) and protein detection such as immunohistochemistry or immunofluorescence to identify both known and unknown cellular markers. In some instances, a biological sample is examined for MYCC protein expression. In some instances, a biological sample is examined for MYCN protein expression. In some instances, a biological sample is examined for Ki67 protein expression. In some instances, a biological sample is examined for Cyclin B1 (CCNB1) protein expression. In some instances, a biological sample is examined for TAF11L1 protein expression. In some instances, a biological sample is examined for SYT1 protein expression. In some instances, a biological sample is examined for SLC12A5 protein expression. In some instances, a biological sample is examined for GABRA1 protein expression. In some instances, a biological sample is examined for EGFR protein expression. In some instances, a biological sample is examined for AKT1 protein expression. In some instances, a biological sample is examined for p65 (RELA) protein expression. In some instances, a biological sample is examined for p50 (NFKB1) protein expression. In some instances, a biological sample is examined for MAPK1 protein expression. In some instances, a biological sample is examined for CXCL8 protein expression. In some instances, a biological sample is examined for CD163 protein expression. In some instances, a biological sample is examined for PROM1 protein expression. In some instances, a biological sample is examined for L1CAM protein expression. In some instances, a biological sample is examined for CD44 protein expression. In some instances, a biological sample is examined for CD68 protein expression.

Without being bound by theory, it is appreciated that the spatial analysis methods described herein can detect a transcript that encodes a protein detected using immunohistochemistry, immunofluorescence (e.g., both Ki67 transcript and protein can be detected in the same biological sample). In addition, because the spatial methods described herein can detect genome wide transcripts, transcripts can be identified that co-localize with a protein such as Ki67. It is appreciated that any protein in the art can be examined in the same experiment as the spatial methods described herein. For instance, in some embodiments, mRNA expression can be determined in one or more analytes selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, and any combination thereof.

Further, in some instances, one can correlate the intensity of detection of the above markers with glioblastoma cancer stage. Gliomas (such as glioblastoma or glioblastoma multiforme) are classified into four grades (I, II, III, and IV). Increases in detection of the above-referenced markers can indicate an increase in grade. Further, given that spatial methods can detect location and abundance of both transcript markers and protein expression, it is possible to identify areas of a glioblastoma sample that comprise tumors of various sizes and metastatic stages. In addition, in some instances, detection of location and abundance of one or more biomarkers can indicate the location of a precursor of a tumor. Further, more advanced tumors can include areas of the sample that resemble precursor or smaller tumors or potential new proliferating regions of tumor cells.

In some embodiments, the thickness of the biological sample (e.g., tissue section), for use in the methods described herein may be dependent on the method used to prepare the sample and the physical characteristics of the tissue. Thus, any suitable section thickness can be used. In some embodiments, the thickness of the biological sample section will be at least 0.1 μm, further preferably at least 0.2, 0.3, 0.4, 0.5, 0.7, 1.0, 1.5, 2, 3, 4, 5, 6, 7, 8, 9, or 10 μm. In some embodiments the thickness of the biological sample section is at least 10, 11, 12, 13, 14, 15, 20, or 30 μm. In some embodiments, the thickness of the biological sample is 5-12 μm.

The biological sample, e.g., tissue sample, can be stained, and imaged prior to, during, and/or after each step of the methods described herein. Any of the methods described herein or known in the art can be used to stain and/or image the biological sample. In some embodiments, the imaging occurs prior to permeabilizing the sample. In some embodiments, the biological sample is stained using a histological staining method, such as hematoxylin and eosin, or H&E stain. In some embodiments, the tissue sample is stained and imaged for about 10 minutes to about 2 hours (or any of the subranges of this range described herein). Additional time may be needed for staining and imaging of different types of biological samples. In some embodiments, the tissue sample is immunofluorescently labelled to identify the presence of particular cellular proteins, such as DAPI for identifying the nucleus of a cell or cells in a tissue.

In some embodiments, the fixed biological samples are sectioned right before use, for example one day prior to use. In some embodiments, the fixed biological sample has been stored on the first substrate in contact with a mounting agent and a coverslip. In some embodiments, the mounting agent comprises glycerin, water-soluble mounting media, or a carbohydrate. In some embodiments, the coverslip is removed prior to the hybridizing of the first probe and the second probe to the analyte. In some embodiments, the fixed biological sample is archived and has been stored on the first substrate for less than four months (e.g., less than one month, less than two months, or less than three months). In some embodiments, the archived fixed biological sample has been stored on the first substrate for at least four months (e.g., at least five months, at least six months, at least seven months, at least eight months, at least nine months, at least ten months, at least eleven months, or at least twelve months). In some embodiments, the archived fixed biological sample has been stored on the first substrate for at least one year (e.g., at least two years, at least three years, at least four years, or at least five years). In some embodiments, the methods disclosed herein provide surprising and unexpected results wherein RNA of sufficient integrity is identified and localized in the fixed biological sample that has been stored for at least four months (e.g., at least five months, at least six months, at least seven months, at least eight months, at least nine months, at least ten months, at least eleven months, at least twelve months, at least two years, at least three years, at least four years, or at least five years).

In some embodiments, the fixed biological sample has been stored on the first substrate at a temperature above −20° C. (e.g., above −18° C., above −16° C., above −14° C., above −12° C., above −10° C., above −8° C., above −6° C., above −4° C., above −2° C., above 0° C., or above 2° C.). In some embodiments, the fixed biological sample has been stored on the first substrate at a temperature above 4° C. (e.g., above 6° C., or above 8° C., above 10° C., or above 12° C., above 14° C., or above 16° C., above 18° C., or above 20° C., above 22° C., or above 24° C., or above 26° C.). In some embodiments, the fixed biological sample has been stored on the first substrate at room temperature, wherein room temperature refers to a temperature around 20-22° C. In some embodiments, the fixed biological sample has been stored on the first substrate at a temperature above room temperature (e.g., above 20° C., above 21° C., or above 22° C., above 23° C., above 24° C., above 25° C., or above 26° C.).

In some embodiments, the paraffin-embedding material is removed (e.g., deparaffinization) from the biological sample (e.g., tissue section) by incubating the biological sample in an appropriate solvent (e.g., xylene), followed by a series of rinses (e.g., ethanol of varying concentrations), and rehydration in water. In some embodiments, the biological sample can be dried following deparaffinization. In some embodiments, after the step of drying the biological sample, the biological sample can be stained (e.g., H&E stain, any of the variety of stains described herein). In some embodiments, after staining the biological sample, the sample can be imaged.

After an FFPE biological sample has undergone deparaffinization, the FFPE biological sample can be further processed. For example, FFPE biological samples can be treated to remove formaldehyde-induced crosslinks (e.g., decrosslinking). In some embodiments, de-crosslinking the formaldehyde-induced crosslinks in the FFPE biological sample can include treating the sample with heat. In some embodiments, decrosslinking the formaldehyde-induced crosslinks can include performing a chemical reaction. In some embodiments, decrosslinking the formaldehyde-induced crosslinks, can include treating the sample with a permeabilization reagent. In some embodiments, decrosslinking the formaldehyde-induced crosslinks can include heat, a chemical reaction, and/or permeabilization reagents.

In some embodiments, decrosslinking formaldehyde-induced crosslinks can be performed in the presence of a buffer. For example, the buffer can be Tris-EDTA (TE) buffer. In some embodiments, the TE buffer has a pH of about 7.0 to about 9.0, about 7.1 to about 8.9, about 7.2 to about 8.8, about 7.3 to about 8.7, about 7.4 to about 8.6, about 7.5 to about 8.5, about 7.6 to about 8.4, about 7.7 to about 8.3, about 7.8 to about 8.2, about 7.9 to about 8.1, or about 8.0.

In some instances, the sample is a human sample. In some instances, the sample is a human breast tissue sample. In some instances, the sample is a human brain tissue sample. In some embodiments, the sample is an embryo sample. The embryo sample can be a non-human embryo sample. In some instances, the sample is a mouse embryo sample.

### (B) Exemplary First and Second Substrates

In some instances, the biological sample is placed (e.g., mounted or otherwise immobilized) on a first substrate. The first substrate can be any solid or semi-solid support upon which a biological sample can be mounted. In some instances, the first substrate is a slide. In some instances, the slide is a glass slide. In some embodiments, the substrate is made of glass, silicon, paper, hydrogel, polymer monoliths, or other material known in the art. In some embodiments, the first substrate (e.g., glass slides) is comprised of an inert material or matrix that has been functionalized by, for example, treating the substrate with a material comprising reactive groups which facilitate mounting of the biological sample.

In some embodiments, the first substrate does not comprise a plurality (e.g., array) of capture probes, each comprising a spatial barcode. In preferred embodiments, if the first substrate does not comprise a plurality of capture probes comprising spatial barcodes, the second substrate does comprises capture probes comprising spatial barcodes, or vice versa.

As an alternative embodiment disclosed herein, in some embodiments, the first substrate comprises a plurality (e.g., array) of capture probes, each comprising a spatial barcode. In this instance, no sandwiching process methods are performed. Instead, ligation of target nucleic acid specific probes are performed solely on the first substrate (i.e., without a second substrate; e.g., without sandwiching process methods). Thus, it is appreciated that methods of capture can be performed on the first substrate as well as the second substrate, and it should be evident throughout the application which embodiment(s) is/are described. Additional disclosure of templated ligation without sandwiching process methods is disclosed in PCT Publ. No. WO 2021/133849 A1 and US Publ. No. US 2021/0285046 A1.

A substrate, e.g., a first substrate and/or a second substrate, can generally have any suitable form or format. For example, a substrate can be flat, curved, e.g., convexly or concavely curved. For example, a first substrate can be curved towards the area where the interaction between a biological sample, e.g., tissue sample, and a first substrate takes place. In some embodiments, a substrate is flat, e.g., planar, chip, or slide. A substrate can contain one or more patterned surfaces within the first substrate (e.g., channels, wells, projections, ridges, divots, etc.).

A substrate, e.g., a first substrate and/or second substrate, can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments wherein a substrate structure is flat, the substrate structure can be any appropriate type of support having a flat surface (e.g., a chip or a slide such as a microscope slide).

First and/or second substrates can optionally include various structures such as, but not limited to, projections, ridges, and channels. A substrate can be micropatterned to limit lateral diffusion of analytes (e.g., to improve resolution of the spatial analysis). A substrate modified with such structures can be modified to allow association of analytes, features (e.g., beads), or probes at individual sites. For example, the sites where a substrate is modified with various structures can be contiguous or non-contiguous with other sites.

In some embodiments, the surface of a first and/or second substrate is modified to contain one or more wells, using techniques such as (but not limited to) stamping, microetching, or molding techniques. In some embodiments in which a first and/or second substrate includes one or more wells, the first substrate can be a concavity slide or cavity slide. For example, wells can be formed by one or more shallow depressions on the surface of the first and/or second substrate. In some embodiments, where a first and/or second substrate includes one or more wells, the wells can be formed by attaching a cassette (e.g., a cassette containing one or more chambers) to a surface of the first substrate structure.

In some embodiments where the first and/or second substrate is modified to contain one or more structures, including but not limited to, wells, projections, ridges, features, or markings, the structures can include physically altered sites. For example, a first and/or second substrate modified with various structures can include physical properties, including, but not limited to, physical configurations, magnetic or compressive forces, chemically functionalized sites, chemically altered sites, and/or electrostatically altered sites. In some embodiments where the first substrate is modified to contain various structures, including but not limited to wells, projections, ridges, features, or markings, the structures are applied in a pattern. Alternatively, the structures can be randomly distributed.

In some embodiments, a first substrate includes one or more markings on its surface, e.g., to provide guidance for aligning at least a portion of the biological sample with a plurality of capture probes on the second substrate during a sandwich process disclosed herein. For example, the first substrate can include a sample area indicator identifying the sample area. In some embodiments, during a sandwiching process described herein the sample area indicator on the first substrate is aligned with an area of the second substrate comprising a plurality of capture probes. In some embodiments, the first and/or second substrate can include a fiducial mark. In some embodiments, the first and/or second substrate does not comprise a fiducial mark. In some embodiments, the first substrate does not comprise a fiducial mark and the second substrate comprises a fiducial mark. Such markings can be made using techniques including, but not limited to, printing, sand-blasting, and depositing on the surface.

In some embodiments, imaging can be performed using one or more fiducial markers, i.e., objects placed in the field of view of an imaging system which appear in the image produced. Fiducial markers are typically used as a point of reference or measurement scale. Fiducial markers can include, but are not limited to, detectable labels such as fluorescent, radioactive, chemiluminescent, and colorimetric labels. The use of fiducial markers to stabilize and orient biological samples is described, for example, in Carter et al., Applied Optics 46:421-427, 2007). In some embodiments, a fiducial marker can be a physical particle (e.g., a nanoparticle, a microsphere, a nanosphere, a bead, a post, or any of the other exemplary physical particles described herein or known in the art).

In some embodiments, a fiducial marker can be present on a first substrate to provide orientation of the biological sample. In some embodiments, a microsphere can be coupled to a first substrate to aid in orientation of the biological sample. In some examples, a microsphere coupled to a first substrate can produce an optical signal (e.g., fluorescence). In some embodiments, a quantum dot can be coupled to the first substrate to aid in the orientation of the biological sample. In some examples, a quantum dot coupled to a first substrate can produce an optical signal.

In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Although not required, it can be advantageous to use a marker that can be detected using the same conditions (e.g., imaging conditions) used to detect a labelled cDNA.

In some embodiments, a fiducial marker can be randomly placed in the field of view. For example, an oligonucleotide containing a fluorophore can be randomly printed, stamped, synthesized, or attached to a first substrate (e.g., a glass slide) at a random position on the first substrate. A tissue section can be contacted with the first substrate such that the oligonucleotide containing the fluorophore contacts, or is in proximity to, a cell from the tissue section or a component of the cell (e.g., an mRNA or DNA molecule). An image of the first substrate and the tissue section can be obtained, and the position of the fluorophore within the tissue section image can be determined (e.g., by reviewing an optical image of the tissue section overlaid with the fluorophore detection). In some embodiments, fiducial markers can be precisely placed in the field of view (e.g., at known locations on a first substrate). In this instance, a fiducial marker can be stamped, attached, or synthesized on the first substrate and contacted with a biological sample. Typically, an image of the sample and the fiducial marker is taken, and the position of the fiducial marker on the first substrate can be confirmed by viewing the image.

In some embodiments, a fiducial marker can be an immobilized molecule (e.g., a physical particle) attached to the first substrate. For example, a fiducial marker can be a nanoparticle, e.g., a nanorod, a nanowire, a nanocube, a nanopyramid, or a spherical nanoparticle. In some examples, the nanoparticle can be made of a heavy metal (e.g., gold). In some embodiments, the nanoparticle can be made from diamond. In some embodiments, the fiducial marker can be visible by eye.

A wide variety of different first substrates can be used for the foregoing purposes. In general, a first substrate can be any suitable support material. Exemplary first substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene polycarbonate, or combinations thereof.

Among the examples of first substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In another example, a first substrate can be a flow cell. Flow cells can be formed of any of the foregoing materials, and can include channels that permit reagents, solvents, features, and analytes to pass through the flow cell. In some embodiments, a hydrogel embedded biological sample is assembled in a flow cell (e.g., the flow cell is utilized to introduce the hydrogel to the biological sample). In some embodiments, a hydrogel embedded biological sample is not assembled in a flow cell. In some embodiments, the hydrogel embedded biological sample can then be prepared and/or isometrically expanded as described herein.

Exemplary substrates similar to the first substrate (e.g., a substrate having no capture probes) and/or the second substrate are described in Section (I) above and in WO 2020/123320.

### (b) Capturing Nucleic Acid Analytes Using Templated Ligation

In some embodiments, the methods, devices, compositions, and systems described herein utilize RNA-templated ligation to detect the analyte. As used herein, spatial “RNA-templated ligation,” or “RTL” or simply “templated ligation” is a process wherein individual probes (e.g., a first probe, a second probe) in a probe pair hybridize to adjacent sequences of an analyte (e.g., an RNA molecule) in a biological sample (e.g., a tissue sample). The RTL probes are then ligated together, thereby creating a ligation product. RNA-templated ligation is disclosed in PCT Publ. No. WO 2021/133849 A1 and US Publ. No. US 2021/0285046 A1.

An advantage to using RTL is that it allows for enhanced detection of low expressing analytes in addition to higher expressing analytes because both probes must hybridize to the analyte in order for the coupling (e.g., ligating) reaction to occur. As used herein, “ligation” refers to an interaction between two probes that results in a single ligation product that comprises the two probes. In some instances, coupling is achieved through ligation. In some instances, coupling is achieved through extension of one probe to the second probe followed by ligation, when the probes hybridized to a target mRNA are not immediately adjacent to each other. In some instances, coupling is achieved through hybridization of a third probe that hybridizes to each of the two probes followed optionally by extension of one probe or gap filling of the sequence between the two probes.

The ligation product that results from the ligation of the two probes serves as a proxy for the target analyte, as such an analyte derived molecule. Further, it is appreciated that probe pairs can be designed to cover any gene of interest. For example, a pair of probes can be designed so that each analyte, e.g., a whole exome, a transcriptome, a genome, can conceivably be detected using a plurality of probe pairs.

In some instances, disclosed herein are methods for analyzing an analyte in a biological sample comprising (a) hybridizing a first probe and a second probe to the analyte, wherein the first probe and the second probe each comprise a sequence that is substantially complementary to adjacent sequences of the analyte, and wherein the second probe comprises a capture probe binding domain; (b) ligating the first probe and the second probe, thereby generating a ligation product comprising the capture probe binding domain; (c) contacting the biological sample with a reagent medium comprising a permeabilization agent and an agent for releasing the ligation product, thereby (i) permeabilizing the biological sample and (ii) releasing the a ligation product from the analyte; and (d) hybridizing the capture probe binding domain of the ligation product to a capture domain of a capture probe, wherein the capture probe comprises: (i) a spatial barcode and (ii) a capture domain.

Also provided herein are methods for analyzing an analyte in a biological sample mounted on a first substrate without an array including (a) hybridizing a first probe and a second probe to the analyte, wherein the first probe and the second probe each include a sequence that is substantially complementary to adjacent sequences of the analyte, and wherein the second probe includes a capture probe binding domain; (b) ligating the first probe and the second probe, thereby generating a ligation product including the capture probe binding domain; (c) aligning the first substrate with a second substrate including an array, such that at least a portion of the biological sample is aligned with at least a portion of the array, wherein the array includes a plurality of capture probes, wherein a capture probe of the plurality of capture probes includes: (i) a spatial barcode and (ii) a capture domain; (d) when the biological sample is aligned with at least a portion of the array, (i) releasing the ligation product from the analyte and (ii) passively or actively migrating the ligation product from the biological sample to the array; and (e) hybridizing the capture probe binding domain of the ligation product to the capture domain.

In some embodiments, transferring the ligated probe from the sample to the capture probe is performed on the first substrate.

In some embodiments, the process of transferring the ligation product from the first substrate to the second substrate is referred to as a “sandwich” process. The sandwich process is described in PCT Patent Application Publication No. WO 2020/123320. Described herein are methods in which an array with capture probes located on a substrate and a biological sample located on a different substrate, are contacted such that the array is in contact with the biological sample (e.g., the substrates are sandwiched together). In some embodiments, the array and the biological sample can be contacted (e.g., sandwiched), without the aid of a substrate holder. In some embodiments, the array and biological sample substrates can be placed in a substrate holder (e.g., an array alignment device) designed to align the biological sample and the array. For example, the substrate holder can have placeholders for two substrates. In some embodiments, an array including capture probes can be positioned on one side of the substrate holder (e.g., in a first substrate placeholder). In some embodiments, a biological sample can be placed on the adjacent side of the substrate holder in a second placeholder. In some embodiments, a hinge can be located between the two substrate placeholders that allows the substrate holder to close, e.g., make a sandwich between the two substrate placeholders. In some embodiments, when the substrate holder is closed the biological sample and the array with capture probes are contacted with one another under conditions sufficient to allow analytes present in the biological sample to interact with the capture probes of the array. For example, dried permeabilization reagents can be placed on the biological sample and rehydrated. A permeabilization solution can be flowed through the substrate holder to permeabilize the biological sample and allow analytes in the biological sample to interact with the capture probes. Additionally, the temperature of the substrates or permeabilization solution can be used to initiate or control the rate of permeabilization. For example, the substrate including the array, the substrate including the biological sample, or both substrates can be held at a low temperature to slow diffusion and permeabilization efficiency. Once sandwiched, in some embodiments, the substrates can be heated to initiate permeabilization and/or increase diffusion efficiency. Transcripts that are released from the permeabilized tissue can diffuse to the array and be captured by the capture probes. The sandwich can be opened, and cDNA synthesis can be performed on the array.

In some embodiments, the methods as disclosed herein include hybridizing of one or more probe pairs (e.g., RTL probes) to adjacent or nearby sequences of a target analyte (e.g., RNA; e.g., mRNA) of interest. In some embodiments, the probe pairs include sequences that are complementary or substantially complementary to an analyte. For example, in some embodiments, each probe includes a sequence that is complementary or substantially complementary to an mRNA of interest (e.g., to a portion of the sequence of an mRNA of interest). In some embodiments, each target analyte includes a first target region and a second target region. In some embodiments, the methods include providing a plurality of first probes and a plurality of second probes, wherein a pair of probes for a target analyte comprises both a first and second probe. In some embodiments, a first probe hybridizes to a first target region of the analyte, and the second probe hybridizes to a second, adjacent or nearly adjacent target region of the analyte.

In some instances, the probes are DNA molecules. In some instances, the first probe is a DNA molecule. In some instances, the second probe is a DNA molecule. In some instances, the first probe comprises at least two ribonucleic acid bases at the 3′ end. In some instances, the second probe comprises a phosphorylated nucleotide at the 5′ end.

RTL probes can be designed using methods known in the art. In some instances, probe pairs are designed to cover an entire transcriptome of a species (e.g., a mouse or a human). In some instances, RTL probes are designed to cover a subset of a transcriptome (e.g., a mouse or a human). In some instances, the methods disclosed herein utilize about 500, about 1000, about 2000, about 3000, about 4000, about 5000, about 6000, about 7000, about 8000, about 9000, about 10,000, about 15,000, about 20,000, or more probe pairs.

In some embodiments, one of the probes of the pair of probes for RTL includes a poly(A) sequence or a complement thereof. In some instances, the poly(A) sequence or a complement thereof is on the 5′ end of one of the probes. In some instances, the poly(A) sequence or a complement thereof is on the 3′ end of one of the probes. In some embodiments, one probe of the pair of probes for RTL includes a degenerate or UMI sequence. In some embodiments, the UMI sequence is specific to a particular target or set of targets. In some instances, the UMI sequence or a complement thereof is on the 5′ end of one of the probes. In some instances, the UMI sequence or a complement thereof is on the 3′ end of one of the probes.

In some instances, the first and second target regions of an analyte are directly adjacent to one another. In some embodiments, the complementary sequences to which the first probe and the second probe hybridize are 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, or about 150 nucleotides away from each other. Gaps between the probes may first be filled prior to coupling (e.g., ligation), using, for example, dNTPs in combination with a polymerase such as polymerase mu, DNA polymerase, RNA polymerase, reverse transcriptase, VENT polymerase, Taq polymerase, and/or any combinations, derivatives, and variants (e.g., engineered mutants) thereof. In some embodiments, when the first and second probes are separated from each other by one or more nucleotides, deoxyribonucleotides are used to extend and couple (e.g., ligate) the first and second probes.

In some instances, the first probe and the second probe hybridize to an analyte on the same transcript. In some instances, the first probe and the second probe hybridize to an analyte on the same exon. In some instances, the first probe and the second probe hybridize to an analyte on different exons. In some instances, the first probe and the second probe hybridize to an analyte that is the result of a translocation event (e.g., in the setting of cancer). The methods provided herein make it possible to identify alternative splicing events, translocation events, and mutations that change the hybridization rate of one or both probes (e.g., single nucleotide polymorphisms, insertions, deletions, point mutations).

In some embodiments, the first and/or second probe as disclosed herein includes at least two ribonucleic acid bases at the 3′ end; a functional sequence; a phosphorylated nucleotide at the 5′ end; and/or a capture probe binding domain. In some embodiments, the functional sequence is a primer sequence.

The “capture probe binding domain” is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or a combination thereof. In some embodiments, the capture probe binding domain includes a random sequence (e.g., a random hexamer or octamer). In some embodiments, the capture probe binding domain is complementary to a capture domain in a capture probe that detects a particular target(s) of interest. In some embodiments, a capture probe binding domain blocking moiety that interacts with the capture probe binding domain is provided. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a ligation product) to a capture probe. In some embodiments, a capture probe binding domain blocking moiety comprises a poly-uridine sequence, a poly-thymidine sequence, or a combination thereof.

Hybridization of the probes to the target analyte can occur at a target having a sequence that is 100% complementary to the probe(s). In some embodiments, hybridization can occur at a target having a sequence that is at least (e.g. at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe(s). After hybridization, in some embodiments, the first probe is extended. After hybridization, in some embodiments, the second probe is extended. For example, in some instances a first probe hybridizes to a target sequence upstream for a second oligonucleotide probe, whereas in other instances a first probe hybridizes to a target sequence downstream of a second probe.

In some embodiments, methods disclosed herein include a wash step after hybridizing the first and the second probes. The wash step removes any unbound oligonucleotides and can be performed using any technique known in the art. In some embodiments, a pre-hybridization buffer is used to wash the sample. In some embodiments, a phosphate buffer is used. In some embodiments, multiple wash steps are performed to remove unbound oligonucleotides. For example, it is advantageous to decrease the amount of unhybridized probes present in a biological sample as they may interfere with downstream applications and methods.

In some embodiments, after hybridization of probes (e.g., first and the second probes) to the target analyte, the probes (e.g., the first probe and the second probe) are coupled (e.g., ligated) together, creating a single ligation product that is complementary to the target analyte. Ligation can be performed enzymatically or chemically, as described herein. For example, the first and second probes are hybridized to the first and second target regions of the analyte, and the probes are subjected to a nucleic acid reaction to ligate them together. For example, the probes may be subjected to an enzymatic ligation reaction using a ligase (e.g., T4 RNA ligase (Rnl2), a Chlorella virus DNA ligase, or a T4 DNA ligase). See, e.g., Zhang L., et al.; Archaeal RNA ligase from thermoccocus kodakarensis for template dependent ligation RNA Biol. 2017; 14(1): 36-44 for a description of KOD ligase. A skilled artisan will understand that various reagents, buffers, cofactors, etc. may be included in a ligation reaction depending on the ligase being used.

In some embodiments, the first probe and the second probes are on a contiguous nucleic acid sequence. In some embodiments, the first probe is on the 3′ end of the contiguous nucleic acid sequence. In some embodiments, the first probe is on the 5′ end of the contiguous nucleic acid sequence. In some embodiments, the second probe is on the 3′ end of the contiguous nucleic acid sequence. In some embodiments, the second probe is on the 5′ end of the contiguous nucleic acid sequence.

In some embodiments, the method further includes hybridizing a third probe to the first probe and the second probe such that the first probe and the second probe abut each other. In some embodiments, the third probe comprises a sequence that is at least 80%, at least 85%, at least 90%, at least 95%, at least 96%, at least 97%, at least 98%, or at least 99% complementary to a portion of the first probe that hybridizes to the third probe. In some embodiments, the third probe comprises a sequence that is 100% complementary to a portion of the first probe that hybridizes to the third probe. In some embodiments, the third probe comprises a sequence that is at least 80%, at least 85%, at least 90%, at least 95%, at least 96%, at least 97%, at least 98%, or at least 99% complementary to a portion of the second probe that hybridizes to the third probe. In some embodiments, the third probe comprises a sequence that is 100% complementary to a portion of the second probe that hybridizes to the third probe.

In some embodiments, a method for identifying a location of an analyte in a biological sample exposed to different permeabilization conditions includes (a) contacting the biological sample with a substrate, wherein the substrate comprises a plurality of capture probes, wherein a capture probe of the plurality of capture probes comprises a capture domain; (b) contacting the biological sample with a first probe and a second probe, wherein the first probe and the second probe are substantially complementary to adjacent sequences of the analyte, and wherein the second probe comprises a capture probe-binding domain that is capable of binding to a capture domain of the capture probe; (c) hybridizing the first probe and the second probe to adjacent sequences of the analyte; (d) coupling (e.g., ligating) the first probe and the second probe, thereby creating a ligation product that is substantially complementary to the analyte; (e) releasing the ligation product from the analyte; (f) hybridizing the capture probe-binding domain of the ligation product to the hybridization domain of the capture probe; (g) hybridizing a padlock oligonucleotide to the ligation product bound to the capture domain (e.g., such that the padlock oligonucleotide is circularized), wherein the padlock oligonucleotide comprises: (i) a first sequence that is substantially complementary to a first portion of the ligation product, (ii) a backbone sequence, and (iii) a second sequence that is substantially complementary to a second portion of the ligation product; and (i) ligating and amplifying the circularized padlock oligonucleotide (e.g., using rolling circle amplification using the circularized padlock oligonucleotide as a template), thereby creating an amplified circularized padlock oligonucleotide, and using the amplified circularized padlock oligonucleotide to identify the location of the analyte in the biological sample.

In some embodiments, the method further includes amplifying the ligation product after the releasing step. In some embodiments, the entire ligation product is amplified. In some embodiments, only part of the ligation product is amplified. In some embodiments, amplification is isothermal. In some embodiments, amplification is not isothermal. Amplification can be performed using any of the methods described herein such as, but not limited to, a strand-displacement amplification reaction, a rolling circle amplification reaction, a ligase chain reaction, a transcription-mediated amplification reaction, an isothermal amplification reaction, and/or a 10 loop-mediated amplification reaction. In some embodiments, amplifying the ligation product creates an amplified ligation product that includes (i) all or part of sequence of the ligation product specifically bound to the capture domain, or a complement thereof, and (ii) all or a part of the sequence of the spatial barcode, or a complement thereof.

In some embodiments, the method further includes determining (i) all or a part of the sequence of the ligation product, or a complement thereof, and (ii) all or a part of the sequence of the spatial barcode, or a complement thereof. In some embodiments, the method further includes using the determined sequence of (i) and (ii) to determine the location and abundance of the analyte in the biological sample.

In some embodiments, after coupling (e.g., ligation) of the first and second probes to create a ligation product, the ligation product is released from the analyte. To release the ligation product, an endoribonuclease (e.g., RNase A, RNase C, RNase H, or RNase I) is used. An endoribonuclease such as RNase H specifically cleaves RNA in RNA:DNA hybrids. In some embodiments, the ligation product is released enzymatically. In some embodiments, an endoribonuclease is used to release the probe from the analyte. In some embodiments, the endoribonuclease is one or more of RNase H. In some embodiments, the RNase H is RNase H1 or RNase H2.

In some embodiments, the releasing of the ligation product includes contacting the biological sample with a reagent medium comprising a permeabilization agent and an agent for releasing the ligation product, thereby permeabilizing the biological sample and releasing the ligation product from the analyte. In some embodiments, the agent for releasing the ligation product comprises a nuclease. In some embodiments, the nuclease is an endonuclease. In some embodiments, the nuclease is an exonuclease. In some embodiments, the nuclease includes an RNase. In some embodiments, the RNase is selected from RNase A, RNase C, RNase H, or RNase I.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In some embodiments, the reagent medium includes a wetting agent.

In some instances, after creation of the ligation product, the methods disclosed herein include simultaneous treatment of the biological sample with a permeabilization agent such as proteinase K (to permeabilize the biological sample) and a releasing agent such as an endonuclease such as RNase H (to release the ligation product from the analyte). In some instances, the permeabilization step and releasing step occur at the same time. In some instances, the permeabilization step occurs before the releasing step. In some embodiments, the permeabilization agent comprises a protease. In some embodiments, the protease is selected from trypsin, pepsin, elastase, or Proteinase K. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin.

In some embodiments, the reagent medium further includes a detergent. In some embodiments, the detergent is selected from sodium dodecyl sulfate (SDS), sarkosyl, saponin, Triton X100™, or Tween-20™. In some embodiments, the reagent medium includes less than 5 w/v % of a detergent selected from sodium dodecyl sulfate (SDS) and sarkosyl. In some embodiments, the reagent medium includes as least 5% w/v % of a detergent selected from SDS and sarkosyl. In some embodiments, the reagent medium does not include SDS or sarkosyl.

In some embodiments, the biological sample and the array are contacted with the reagent medium for about 1 to about 60 minutes (e.g., about 1 to about 55 minutes, about 1 to about 50 minutes, about 1 to about 45 minutes, about 1 to about 40 minutes, about 1 to about 35 minutes, about 1 to about 30 minutes, about 1 to about 25 minutes, about 1 to about 20 minutes, about 1 to about 15 minutes, about 1 to about 10 minutes, about 1 to about 5 minutes, about 5 to about 60 minutes, about 5 to about 55 minutes, about 5 to about 50 minutes, about 5 to about 45 minutes, about 5 to about 40 minutes, about 5 to about 35 minutes, about 5 to about 30 minutes, about 5 to about 25 minutes, about 5 to about 20 minutes, about 5 to about 15 minutes, about 5 to about 10 minutes, about 10 to about 60 minutes, about 10 to about 55 minutes, about 10 to about 50 minutes, about 10 to about 45 minutes, about 10 to about 40 minutes, about 10 to about 35 minutes, about 10 to about 30 minutes, about 10 to about 25 minutes, about 10 to about 20 minutes, about 10 to about 15 minutes, about 15 to about 60 minutes, about 15 to about 55 minutes, about 15 to about 50 minutes, about 15 to about 45 minutes, about 15 to about 40 minutes, about 15 to about 35 minutes, about 15 to about 30 minutes, about 15 to about 25 minutes, about 15 to about 20 minutes, about 20 to about 60 minutes, about 20 to about 55 minutes, about 20 to about 50 minutes, about 20 to about 45 minutes, about 20 to about 40 minutes, about 20 to about 35 minutes, about 20 to about 30 minutes, about 20 to about 25 minutes, about 25 to about 60 minutes, about 25 to about 55 minutes, about 25 to about 50 minutes, about 25 to about 45 minutes, about 25 to about 40 minutes, about 25 to about 35 minutes, about 25 to about 30 minutes, about 30 to about 60 minutes, about 30 to about 55 minutes, about 30 to about 50 minutes, about 30 to about 45 minutes, about 30 to about 40 minutes, about 30 to about 35 minutes, about 35 to about 60 minutes, about 35 to about 55 minutes, about 35 to about 50 minutes, about 35 to about 45 minutes, about 35 to about 40 minutes, about 40 to about 60 minutes, about 40 to about 55 minutes, about 40 to about 50 minutes, about 40 to about 45 minutes, about 45 to about 60 minutes, about 45 to about 55 minutes, about 45 to about 50 minutes, about 50 to about 60 minutes, about 50 to about 55 minutes, or about 55 to about 60 minutes). In some embodiments, the biological sample and the array are contacted with the reagent medium for about 30 minutes.

In some embodiments, the ligation product includes a capture probe binding domain, which can hybridize to a capture probe (e.g., a capture probe immobilized, directly or indirectly, on a substrate). In some embodiments, the capture probe includes a spatial barcode and the capture domain. In some embodiments, the capture probe binding domain of the ligation product specifically binds to the capture domain of the capture probe.

In some embodiments, methods provided herein include mounting a biological sample on a first substrate, then aligning the first substrate with a second substrate including an array, such that at least a portion of the biological sample is aligned with at least a portion of the array, wherein the array includes a plurality of capture probes. After hybridization of the ligation product to the capture probe, downstream methods as disclosed herein can be performed.

In some embodiments, at least 50% of ligation products released from the portion of the biological sample aligned with the portion of the array are captured by capture probes of the portion of the array. In some embodiments, at least 50%, at least 55%, at least 60%, at least 65%, at least 70%, at least 75%, at least 85%, at least 90%, at least 95%, at least 96%, at least 97%, at least 98%, at least 99%, or 100% of ligation products are detected in spots directly under the biological sample.

In some embodiments, the capture probe includes a poly(T) sequence. In some embodiments, capture probe includes a sequence specific to the analyte. In some embodiments, the capture probe includes a functional domain. In some embodiments, the capture probe further includes one or more functional domains, a unique molecular identifier (UMI), a cleavage domain, and combinations thereof. In some embodiments, the capture probe binding domain includes a poly(A) sequence. In some embodiments, the capture probe binding domain includes a sequence complementary to a capture domain of a capture probe that detects a target analyte of interest. In some embodiments, the analyte is RNA. In some embodiments, the analyte is mRNA.

In some embodiments, the ligation product (e.g., the analyte derived molecule) includes a capture probe binding domain, which can hybridize to a capture probe (e.g., a capture probe immobilized, directly or indirectly, on a substrate). Methods provided herein include contacting a biological sample with a substrate, wherein the capture probe is affixed to the substrate (e.g., immobilized to the substrate, directly or indirectly). After hybridization of the ligation product to the capture probe, downstream methods as disclosed herein (e.g., sequencing, in situ analysis such as RCA) can be performed.

An exemplary embodiment of a workflow for analysis of protein and RNA analytes is provided herein. A fixed tissue sample mounted on a first substrate (e.g., a slide-mounted tissue sample) is decrosslinked, followed by hybridization of probe pairs to nucleic acid analyte analytes. A first and second probe of a probe pair is ligated. The sample is optionally washed (e.g., with a buffer). In some embodiments, the ligation products and antibody oligonucleotide tags are released from the tissue under sandwich conditions as described herein. For the sandwich conditions, the tissue-mounted slide can be aligned with an array and permeabilized with a reagent medium in the sandwich configuration as described herein. In some embodiments, the reagent medium comprises RNase and a permeabilization agent (e.g., Proteinase K). Permeabilization releases the ligation product and capture agent barcode domain, for capture onto a second substrate comprising an array with a plurality of capture probes. After capture of the ligation product and capture agent barcode domain, the tissue slide can be removed (e.g., the sandwich can be “opened” or “broken”).

In some embodiments, following opening of the sandwich (if sandwich methods are used), the capture probes can be extended, sequencing libraries can be prepared and sequenced, and the results can be analyzed computationally. Methods of analyte analysis is disclosed in PCT Publ. No. WO 2021/133849 A1 and US Publ. No. US 2021/0285046 A1.

In some embodiments, the method further includes determining (i) all or part of the sequence of the capture agent barcode domain; and (ii) all or a part of the sequence of the spatial barcode, or a complement thereof. In some embodiments, the method further includes using the determined sequence of (i), and (ii) to analyze the different analyte in the biological sample. In some embodiments, the releasing step further releases the capture agent barcode domain from the different analyte. In some embodiments, the different analyte is a protein analyte. In some embodiments, the protein analyte is an extracellular protein. In some embodiments, the protein analyte is an intracellular protein.

### (c) Substrate Sandwich Transfer Processes

In some embodiments, one or more analytes from the biological sample are released from the biological sample and migrate to a substrate comprising an array of capture probes for attachment to the capture probes of the array. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the alignment of the first substrate and the second substrate is facilitated by a sandwiching process. Accordingly, described herein are methods, compositions, devices, and systems for sandwiching together the first substrate as described herein with a second substrate having an array with capture probes.

During the exemplary sandwiching process, the first substrate is aligned with the second substrate, such that at least a portion of the biological sample is aligned with at least a portion of the array (e.g., aligned in a sandwich configuration). As shown, the second substrate is in a superior position to the first substrate. In some embodiments, the first substrate may be positioned superior to the second substrate. In some embodiments, the first and second substrates are aligned to maintain a gap or separation distance between the two substrates. When the first and second substrates are aligned, one or more analytes are released from the biological sample and actively or passively migrate to the array for capture. In some embodiments, the migration occurs while the aligned portions of the biological sample and the array are contacted with a reagent medium. The released one or more analytes may actively or passively migrate across the gap via the reagent medium toward the capture probes, and be captured by the capture probes.

In some embodiments, the separation distance between first and second substrates is maintained between 2 microns and 1 mm (e.g., between 2 microns and 800 microns, between 2 microns and 700 microns, between 2 microns and 600 microns, between 2 microns and 500 microns, between 2 microns and 400 microns, between 2 microns and 300 microns, between 2 microns and 200 microns, between 2 microns and 100 microns, between 2 microns and 25 microns, between 2 microns and 10 microns), measured in a direction orthogonal to the surface of first substrate that supports sample. In some embodiments, the separation distance 2307 between first and second substrates is less than 50 microns. In some instances, the distance is 2 microns. In some instances, the distance is 2.5 microns. In some instances, the distance is about 2, 3, 4, 5, 6, 7, 8, 9, 10, 11, 12, 13, 14, 15, 16, 17, 18, 19, 20, 21, 22, 23, 24, or 25 microns. In some embodiments, second substrate is placed in direct contact with the sample on the first substrate ensuring no diffusive spatial resolution losses. In some embodiments, the separation distance is measured in a direction orthogonal to a surface of the first substrate that supports the biological sample.

In some embodiments, the sandwiching process may be facilitated by a device, sample holder, sample handling apparatus, or system described in, e.g., US. Patent Application Pub. No. 20210189475, PCT/US2021/036788, or PCT/US2021/050931.

In some embodiments, the first and second substrates are placed in a substrate holder (e.g., an array alignment device) configured to align the biological sample and the array. In some embodiments, the device comprises a sample holder. In some embodiments, the sample holder includes a first member and a second member that receive a first substrate and a second substrate, respectively. The device can include an alignment mechanism that is connected to at least one of the members and aligns the first and second members. Thus, the devices of the disclosure can advantageously align the first substrate and the second substrate and any samples, barcoded probes, or permeabilization reagents that may be on the surface of the first and second substrates.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the analyte from the biological sample.

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The alignment mechanism may be configured to move the second member along the axis orthogonal to the second plane and/or move the first member along an axis orthogonal to the first plane.

In some embodiments, the alignment mechanism includes a linear actuator. In some embodiments, the alignment mechanism includes one or more of a moving plate, a bushing, a shoulder screw, a motor bracket, and a linear actuator. The moving plate may be coupled to the first member or the second member. The alignment mechanism may, in some cases, include a first moving plate coupled to the first member and a second moving plate coupled to the second member. In some embodiments, the linear actuator is configured to move the second member along an axis orthogonal to the plane of the first member and/or the second member. For example, the moving plate may be coupled to the second member and adjust the separation distance along a z axis (e.g., orthogonal to the second substrate) by moving the moving plate up in a superior direction toward the first substrate. In some embodiments, the linear actuator is configured to move the first member along an axis orthogonal to the plane of the first member and/or the second member. The movement of the moving plate may be accomplished by the linear actuator configured to move the first member and/or the second member at a velocity. The velocity may be controlled by a controller communicatively coupled to the linear actuator. In some embodiments, the linear actuator is configured to move the first member, the second member, or both the first member and the second member at a velocity of at least 0.1 mm/sec (e.g., at least 0.1 mm/sec to 2 mm/sec). In some aspects, the velocity may be selected to reduce or minimize bubble generation or trapping within the reagent medium. In some embodiments, the linear actuator is configured to move the first member, the second member, or both the first member and the second member with an amount of force of at least 0.1 pounds (e.g., between 0.1-4.0 pounds of force).

In some aspects, the velocity of the moving plate (e.g., closing the sandwich) may affect bubble generation or trapping within the reagent medium. It may be advantageous to minimize bubble generation or trapping within the reagent medium during the “sandwiching” process, as bubbles can interfere with the migration of analytes through the reagent medium to the array. In some embodiments, the closing speed is selected to minimize bubble generation or trapping within the reagent medium. In some embodiments, the closing speed is selected to reduce the time it takes the flow front of the reagent medium from an initial point of contact with the first and second substrate to sweep across the sandwich area (also referred to herein as “closing time”). In some embodiments, the closing speed is selected to reduce the closing time to less than about 1100 milliseconds (ms). In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less.

Further details on support devices, sample holders, sample handling apparatuses, or systems for implementing a sandwiching process are described in, e.g., PCT Publ. No. WO 2021/0189475 and PCT/US2021/050931.

Analytes within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein)

Provided herein are methods for delivering a fluid to a biological sample disposed on an area of a first substrate and an array disposed on a second substrate.

In some aspects, it may be possible to reduce or eliminate bubble formation between the slides using a variety of filling methods and/or closing methods.

Workflows described herein may include contacting a drop of the reagent medium (e.g., liquid reagent medium, e.g., a permeabilization solution) disposed on a first substrate or a second substrate with at least a portion of the second substrate or first substrate, respectively. In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In some embodiments, the one or more spacers is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

Further details on angled closure workflows, and devices and systems for implementing an angled closure workflow, are described in PCT/US2021/036788 and PCT/US2021/050931.

Additional configurations for reducing or eliminating bubble formation, and/or for reducing unwanted fluid flow, are described in PCT/US2021/036788

In some embodiments, the reagent medium comprises a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). Exemplary permeabilization reagents are described in in PCT Patent Application Publication No. WO 2020/123320

In some embodiments, the reagent medium comprises a protease. Exemplary proteases include, e.g., pepsin, trypsin, pepsin, elastase, and proteinase K. Exemplary proteases are described in PCT Patent Application Publication No. WO 2020/123320.

In some embodiments, the reagent medium comprises a detergent. Exemplary detergents include sodium dodecyl sulfate (SDS), sarkosyl, saponin, Triton X-100™, and Tween-20™. Exemplary detergents are described in PCT Patent Application Publication No. WO 2020/123320.

In some embodiments, the reagent medium comprises a nuclease. In some embodiments, the nuclease comprises am RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNAse, and a sodium salt thereof.

The sample holder is compatible with a variety of different schemes for contacting the aligned portions of the biological sample and array with the reagent medium to promote analyte capture. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some instances, the aligned portions of the biological sample and the array are in contact with the reagent medium for about 1 minute. In some instances, the aligned portions of the biological sample and the array are in contact with the reagent medium for about 5 minutes. In some instances, the aligned portions of the biological sample and the array are in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes. In some instances, the aligned portions of the biological sample and the array are in contact with the reagent medium for about 30 minutes.

In some embodiments, following initial contact between sample and a permeabilization agent, the permeabilization agent can be removed from contact with sample (e.g., by opening sample holder) before complete permeabilization of sample. For example, in some embodiments, only a portion of sample is permeabilized, and only a portion of the analytes in sample may be captured by feature array. In some instances, the reduced amount of analyte captured and available for detection can be offset by the reduction in lateral diffusion that results from incomplete permeabilization of sample. In general, the spatial resolution of the assay is determined by the extent of analyte diffusion in the transverse direction (i.e., orthogonal to the normal direction to the surface of sample). The larger the distance between the sample on the first substrate and the feature array on the second substrate, the greater the extent of diffusion in the transverse direction, and the concomitant loss of resolution. Analytes liberated from a portion of the sample closest to the feature array have a shorter diffusion path, and therefore do not diffuse as far laterally as analytes from portions of the sample farthest from the feature array. As a result, in some instances, incomplete permeabilization of the sample (by reducing the contact interval between the permeabilization agent and the sample) can be used to maintain adequate spatial resolution in the assay.

In some instances, the device is configured to control a temperature of the first and second substrates. In some embodiments, the temperature of the first and second members is lowered to a first temperature that is below room temperature (e.g., 25 degrees Celsius) (e.g., degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes that are released from the permeabilized tissue of the sample diffuse to the surface of the second substrate and are captured on the array (e.g., barcoded probes) of the second substrate. In a fourth step, the first substrate and the second substrate are separated (e.g., pulled apart) and temperature control is stopped.

In some embodiments, where either the first substrate or substrate second (or both) includes wells, a permeabilization solution can be introduced into some or all of the wells, and then the sample and the feature array can be contacted by closing the sample holder to permeabilize the sample. In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of analytes from the sample to the array.

In certain embodiments, different permeabilization agents or different concentrations of permeabilization agents can be infused into array features (e.g., beads) or into a hydrogel layer as described above. By locally varying the nature of the permeabilization reagent(s), the process of analyte capture from the sample can be spatially adjusted.

In some instances, migration of the analyte from the biological sample to the second substrate is passive (e.g., via diffusion). Alternatively, in certain embodiments, migration of the analyte from the biological sample is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher analyte capture efficiency and better spatial fidelity of captured analytes (e.g., on a feature array) than random diffusion onto matched substrates without the application of an electric field (e.g., via manual alignment of the two substrates). Exemplary methods of electrophoretic migration are described in WO 2020/176788.

Loss of spatial resolution can occur when analytes migrate from the sample to the feature array and a component of diffusive migration occurs in the transverse (e.g., lateral) direction, approximately parallel to the surface of the first substrate on which the sample is mounted. To address this loss of resolution, in some embodiments, a permeabilization agent deposited on or infused into a material with anisotropic diffusion can be applied to the sample or to the feature array. The first and second substrates are aligned by the sample holder and brought into contact. A permeabilization layer that includes a permeabilization solution infused into an anisotropic material is positioned on the second substrate.

In some embodiments, the feature array can be constructed atop a hydrogel layer infused with a permeabilization agent. The hydrogel layer can be mounted on the second substrate, or alternatively, the hydrogel layer itself may function as the second substrate. When the first and second substrates are aligned, the permeabilization agent diffuses out of the hydrogel layer and through or around the feature array to reach the sample. Analytes from the sample migrate to the feature array. Direct contact between the feature array and the sample helps to reduce lateral diffusion of the analytes, mitigating spatial resolution loss that would occur if the diffusive path of the analytes was longer.

Spatial analysis workflows can include a sandwiching process described herein. In some embodiments, the workflow includes provision of the first substrate comprising the biological sample. In some embodiments, the workflow includes, mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

The biological sample can be stained using known staining techniques, including, without limitation, Can-Grunwald, Giemsa, hematoxylin and eosin (H&E), hematoxylin, Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes biological staining using hematoxylin. In some embodiments, staining includes identifying analytes using fluorescently-conjugated antibodies, e.g., by immunofluorescence. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample. In some instances, a biological sample on the first substrate is stained.

In some instances, methods for immunofluorescence include a blocking step. The blocking step can include the use of blocking probes to decrease unspecific binding of the antibodies. The blocking step can optionally further include contacting the biological sample with a detergent. In some instances, the detergent can include Triton X-100™. The method can further include an antibody incubation step. In some embodiments, the antibody incubation step effects selective binding of the antibody to antigens of interest in the biological sample. In some embodiments, the antibody is conjugated to an oligonucleotide (e.g., an oligonucleotide-antibody conjugate as described herein). In some embodiments, the antibody is not conjugated to an oligonucleotide. In some embodiments, the method further comprises an antibody staining step. The antibody staining step can include a direct method of immunostaining in which a labelled antibody binds directly to the analyte being stained for. Alternatively, the antibody staining step can include an indirect method of immunostaining in which a first antibody binds to the analyte being stained for, and a second, labelled antibody binds to the first antibody. In some embodiments, the antibody staining step is performed prior to sandwich assembly. In some embodiments wherein an oligonucleotide-antibody conjugate is used in the antibody incubation step, the method does not comprise an antibody staining step.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, image are captured using high resolution techniques (e.g., having 300 dots per square inch (dpi) or greater). For example, images can be captured using brightfield imaging (e.g., in the setting of hematoxylin or H&E stain), or using fluorescence microscopy to detect adhered labels. In some instances, high resolution images are captured temporally using e.g., confocal microscopy. In some instances, a low resolution image is captured. A low resolution image (e.g., images that are about 72 dpi and normally have an RGB color setting) can be captured at any point of the workflow, including but not limited to staining, destaining, permeabilization, sandwich assembly, and migration of the analytes. In some instances, a low resolution image is taken during permeabilization of the biological sample.

In some embodiments, the location of the one or more analytes in a biological sample are determined by immunofluorescence. In some embodiments, one or more detectable labels (e.g., fluorophore-labeled antibodies) bind to the one or more analytes that are captured (hybridized to) by a probe on the first slide and the location of the one or more analytes is determined by detecting the labels under suitable conditions. In some embodiments, one or more fluorophore-labeled antibodies are used to conjugate to a moiety that associates with a probe on the first slide or the analyte that is hybridized to the probe on the first slide. In some instances, the location(s) of the one or more analytes is determined by imaging the fluorophore-labeled antibodies when the fluorophores are excited by a light of a suitable wavelength. In some embodiments, the location of the one or more analytes in the biological sample is determined by correlating the immunofluorescence data to an image of the biological sample. In some instances, the tissue is imaged throughout the permeabilization step.

In some instances, the biological samples can be destained. In some instances, destaining occurs prior to permeabilization of the biological sample. By way of example only, H&E staining can be destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

In some instances, after the sandwiching process the first substrate and the second substrate are separated (e.g., such that they are no longer aligned in a sandwich configuration, also referred to herein as opening the sandwich). In some embodiments, subsequent analysis (e.g., cDNA synthesis, library preparation, and sequences) can be performed on the captured analytes after the first substrate and the second substrate are separated.

In some embodiments, the process of transferring the ligation product or methylated-adaptor-containing nucleic acid from the first substrate to the second substrate is referred to interchangeably herein as a “sandwich process,” “sandwiching process,” or “sandwiching”. The sandwich process is further described in PCT Patent Application Publication No. WO 2020/123320, PCT/US2021/036788, and PCT/US2021/050931.

### (d) Multiplexing the Sandwich Processes

This disclosure also provides methods, compositions, devices, and systems for using a single capture probe-containing to detect analytes from different biological samples (e.g., tissues) on different slides using serial sandwich processes. Thus, only one capture probe-containing array is necessary for the methods disclosed herein. In this way, as described herein, analytes from different samples or tissues can be captured serially and demultiplexed by sample-specific index sequences.

The methods include generating a ligation product in multiple biological samples (i.e., a first sample, a second sample, a third sample, etc.). Generation of a ligation product has been described above, and, the same methods are used herein to generate a ligation product from analytes that are either protein analytes or nucleic acid (i.e., mRNA) analytes. That is, in some instances, the multiplexed methods disclosed herein can be used to detect protein analytes. In other instances, the multiplexed methods disclosed herein can be used to detect nucleic acid (i.e., mRNA) analytes.

Exemplary fiducial markers are described in PCT Patent Application Publication No. WO 2020/123320.

Such methods, compositions, devices, and systems allow for detection of analytes in multiple samples using only one gene expression slide and can be performed on any slide sample described herein. For example, in some instances, the biological sample is a formalin-fixed, paraffin-embedded (FFPE) sample, a frozen sample, or a fresh sample. In some instances, the biological sample is contacted with one or more stains. In some instances, the one or more stains include hematoxylin and eosin. In some instances, cell markers are detected using methods known in the art (e.g., using one or more optical labels such as fluorescent labels, radioactive labels, chemiluminescent labels, calorimetric labels, or colorimetric labels. In some instances, the biological sample is imaged before generating a ligation product and before transferring the ligation product to the gene expression slide.

The multiplex methods, compositions, devices, and systems described herein allow for detection of different types of samples and different analytes. For example in some instances, the samples used in the multiplex sandwich methods are from different species. In some instances, the samples used in the multiplex sandwich methods are from the same species but different individuals in the same species. In some instances, the samples used in the multiplex sandwich methods are from the same individual organism. In some instances, the samples are from different tissues or cell types. In some instances, the samples are from the same tissues or cell types. In some instances, the samples are from the same subject taken at different time points (e.g., before and after treatment). It is appreciated that the samples can be from any source so long as ligated products having sample index sequences unique to each sample are generated.

Multiple samples can be used in the methods described herein. For example, in some instances, at least two samples are used. In some instances, more than two samples (e.g., at least 3, 4, 5, 6, 7, 8, 9, 10, or more) samples are used in the methods disclosed herein. It is appreciated that each sample can be from different sources (e.g., different species, different organisms). In some embodiments, from each sample, the same gene is detected and identified. In some embodiments, for each sample, different genes are detected and identified.

In order to differentiate one sample from another, probe for each sample in a multiplexed setting can include one or more unique sequences to identify the origin of the ligation product. In some instances, the unique sequence is a sample index sequence. In some instances, probes for each sample include one or more (e.g., at least 1, 2, 3, 4, 5, or more) unique sample index sequence to identify the origin of the ligation product.

In some instances, the sample index is about 5 nucleotides to about 50 nucleotides long (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotides long. In some embodiments, the sample index is about 5-15 nucleotides long. In some embodiments, the sample index is about 10-12 nucleotides long. Both synthetic and/or naturally-occurring nucleotides can be used to generate a sample index sequence. It is appreciated that any sequence can be designed so long as it is unique among other sample index sequences and optionally that it can be distinguished from any sequence in the genome of the sample.

A sample index sequence can be located anywhere on the ligation product so long as it does not affect (1) hybridization of the probes to the analyte, (2) ligation of the probes to generate the ligation product, and (3) hybridization of the capture probe binding domain to the capture probe on an array. For example, in some instances, the sample index sequence can be located on the first probe (e.g., the left hand probe). In some instances, the sample index is located on the flap of the first probe that does not hybridize to the analyte. In some instances, the sample index sequence can be located on the second probe (e.g., the right hand probe). In some instances, the sample index is located on the flap of the second probe that does not hybridize to the analyte.

### (e) Systems and Kits

Also disclosed herein are systems and kits used for any one of the methods disclosed herein. In some instances, the system of kit is used for analyzing an analyte in a glioblastoma biological sample. In some instances, the system or kit includes a support device configured to retain a first substrate and a second substrate, wherein the biological sample is placed on the first substrate, and wherein the second substrate comprises a plurality of capture probes, wherein a capture probe of the plurality of capture probes comprises: (i) a spatial barcode and (ii) a capture domain. In some instances, the system or kit includes a first probe and a second probe, wherein the first probe and the second probe each comprise a sequence that is substantially complementary to adjacent sequences of the analyte, wherein the second probe comprises a capture probe binding domain, and wherein the first probe and the second probe are capable of being ligated together to form a ligation product. In some instances, the system or kit further includes a reagent medium comprising a permeabilization agent and optionally an agent for releasing the ligation product. In some instances, the system or kit includes instructions for performing any one of the methods described herein.

In some instances, the permeabilization agent is pepsin or proteinase K. In some instances, the agent for releasing the ligation product is an RNAse, optionally wherein the RNAse is RNAse H.

In some instances, the system or kit further includes an alignment mechanism on the support device to align the first substrate and the second substrate. In some instances, the alignment mechanism comprises a linear actuator and the first substrate comprises a first member and the second substrate comprises a second member. The linear actuator can be configured to move the second member along an axis orthogonal to the plane or the first member and/or the second member. The linear actuator can be configured to move the first member along an axis orthogonal to the plane of the first member and/or the second member. The linear actuator can be configured to move the first member, the second member, or both the first member and the second member at a velocity of at least 0.1 mm/sec. Finally, in some instances, the linear actuator can be configured to move the first member, the second member, or both the first member and the second member with an amount of force of at least 0.1 lbs.

### EXAMPLES

### Example 1—Spatial Gene Expression Analysis of FFPE-Fixed Samples Using Templated Ligation

As a non-limiting example, templated ligation on an FFPE-fixed sample can be performed. Briefly, FFPE-fixed samples are deparaffinized, stained (e.g., H&E stain), and imaged. Samples are destained (e.g., using HCl) and decrosslinked. Following decrosslinking, samples treated with pre-hybridization buffer (e.g., hybridization buffer without the first and second probes), probes are added to the sample, probes hybridized, and samples are washed. Ligase is added to the samples to ligate hybridized probes to generate a ligation product and samples are then washed. Probes are released from the analyte by contacting the biological sample with RNAse H. Samples are permeabilized to facilitate capture of the ligation product by the capture probes on the substrate. Ligation products that hybridize to the capture probes are extended. The extended capture probes are denatured. Denatured, extended capture probes are indexed and, and the amplified libraries are subjected to quality control before being sequenced.

Additional examples of templated ligation are disclosed in PCT Publ. No. WO 2021/133849 A1 and US Publ. No. US 2021/0285046 A1.

### Example 2—Methods for Capturing Ligation Products from Glioblastoma Tissue Sections Using a Sandwich Transfer Process

In a non-limiting example, FFPE sections of human glioblastoma tissue on standard slides (for sandwich conditions) that were recently sectioned were used in an experiment over the course of a few days.

To determine the gene expression spatial profiles of mRNA in FFPE tissue samples, microtome sections of FFPE tissues were placed on slides and dried for a few hours until the sections become transparent prior to drying overnight in a desiccator. Deparaffinization of the dried tissue sections was performed by baking the slides with coverslips at 60° C. followed by incubating the tissues in xylene and decreasing ethanol washes, finally washing the deparaffinized tissue sections prior to staining. H&E staining was performed on the tissue sections as known in the art.

Following imaging, the tissue sections were destained on a thermocycler by the addition a 0.1 N HCl solution to the tissue sections, incubating at 42° C. for 15 min., followed by addition of citrate in a PBS-Tween buffer at pH 6.0 at 95° C. for 60 min. for tissue decrosslinking.

After decrosslinking of the tissue sections, hybridization probes were added to the tissue sections. For each nucleic acid target, two probes that hybridize to adjacent sequences were used to target a nucleic acid. The PBS-T buffered solution was removed and a FFPE hybridization solution comprising 2.4 nM each of the two probes was added to the tissue sections and incubated overnight at 50° C.

Concurrent with the gene expression spatial mRNA workflow described in this example, protein expression profiles were determined via labelling of protein targets in tissue sections. On the same tissue sections on slides, following the decrosslinking steps the tissue sections were exposed concurrently with immunofluorescence labeling reagents using methods known in the art.

After an overnight incubation, the tissue sections were washed several times with a post-hybridization wash solution that includes SSC, at 50° C. for around 5 min. for each wash. The wash solution was removed and the two probes that are hybridized to the target nucleic acid sequences were ligated together by addition of a ligation mix to the tissue sections and incubation at 37° C. for 1 hr. The tissue sections were washed several times by adding ligation wash buffer and incubating the tissue sections at 57° C. for 5 min. A final wash of the tissue sections in SSC solution at 57° C. for 5 min prior to room temperature was performed.

Post probe ligation, the ligation products from the standard slide were transferred via the sandwich method to a second slide that contained an array including capture probes that contained spatial barcodes and capture domains. The sandwich transfer was performed as described previously, also as described in PCT/US2021/061401.

Following transfer of the ligation products from the standard slide to the spatial array slide, captured ligation products were used as templates for generating expression products from the capture probes and for completing their extension to include the sequences of the capture probe, including the spatial barcode. Sequencing libraries were prepared, sequenced, and the results were analyzed computationally.

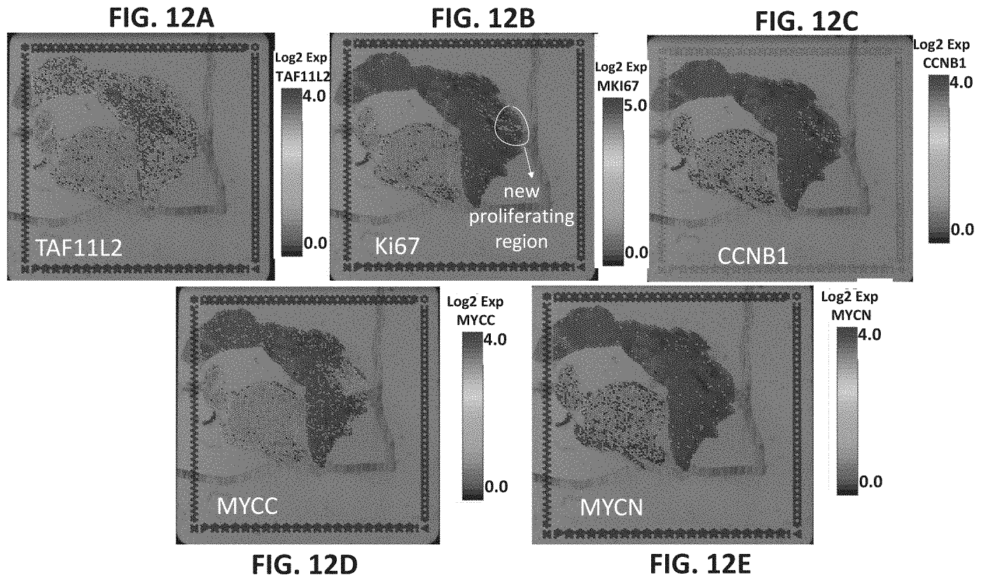

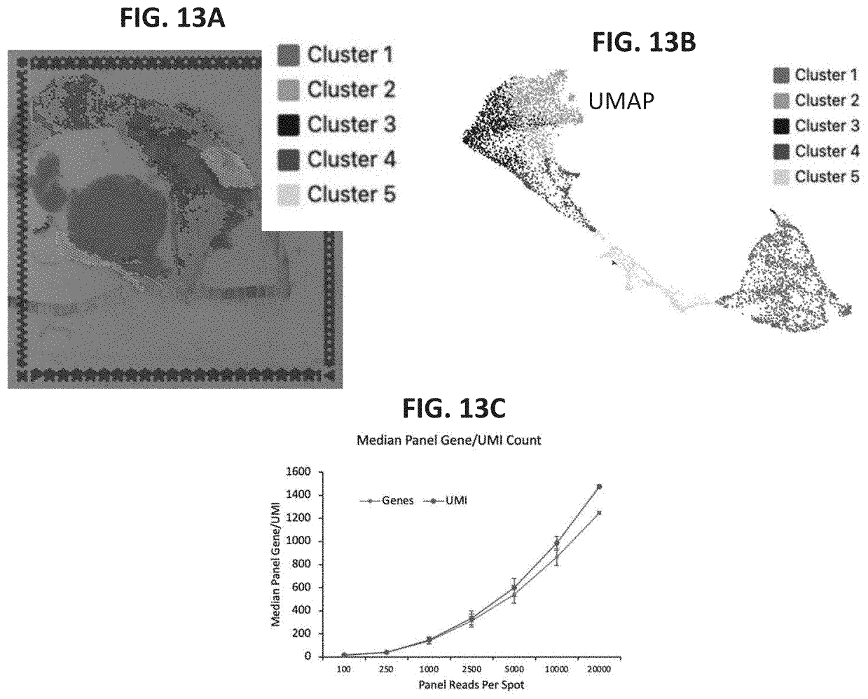

Using the above protocol, glioblastoma sections were examined. Ligated paired probes that were transferred from the original standard slide to the array slide comprising capture probes using sandwiching process methods detected similar median panel gene counts and median unique molecule identifier counts per spot, suggesting consistent mRNA target detection throughout the sample. FIG. 13C. Further, the entire sample readily transcribed TAF11L2, demonstrating consistent detection of an analyte in the entire sample. FIG. 12A.

Transcriptome analysis from the two glioblastoma samples identified a large mass of cells expressing high levels of oncogenes MYCC (FIG. 12D), MYCN (FIG. 12E), and cell cycle related genes Ki67 (FIG. 12B) and CCNB1 (FIG. 12C). In addition, the sandwiching methods used herein enabled identification of a smaller mass of cells which expressed high levels of MYCC and weak Ki67. Both tumors segregated as distinct clusters (FIGS. 13A-13B), however, the bigger tumor contained a small population of cells that clustered with smaller tumor, indicative of cells which are precursors to the smaller tumor. Further, regions of Ki67 expression (shown in FIG. 12B) indicate a region of proliferation that has been newly identified.

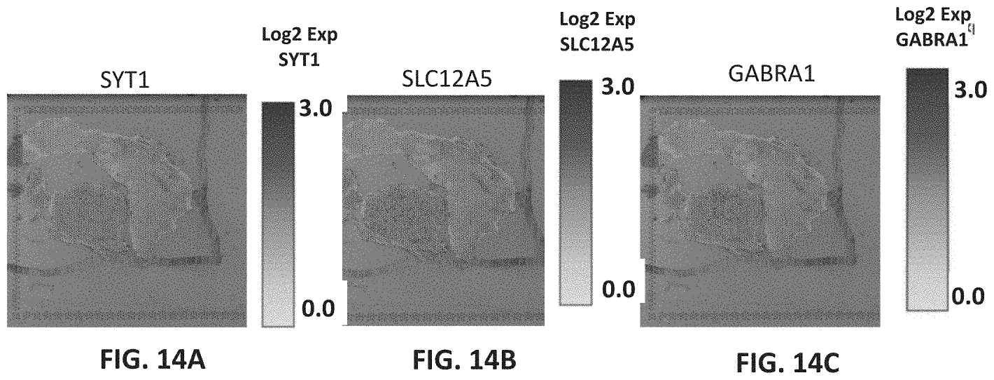

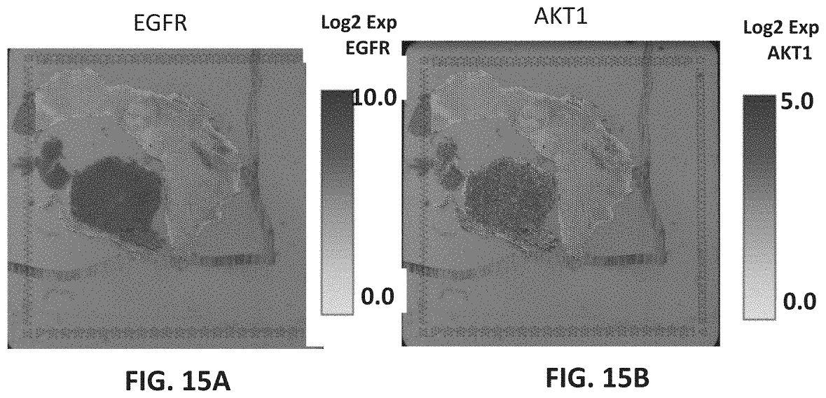

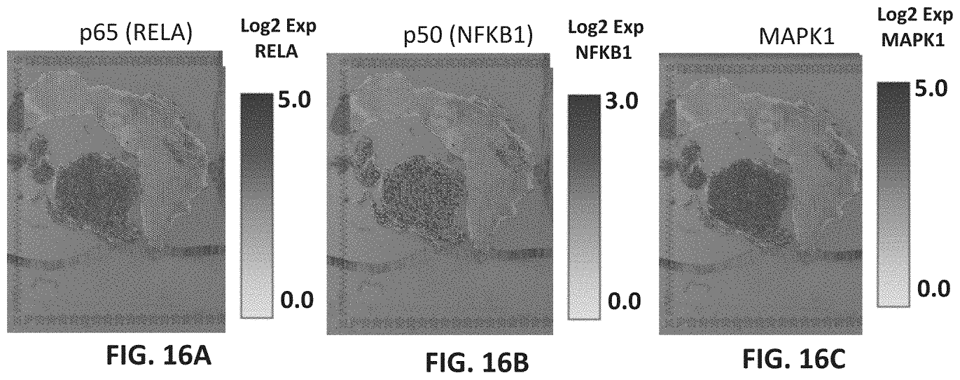

Molecular characterization of the tumor showed that the tumor expressed low levels of proneural type markers such as SYT1 (FIG. 14A), SLC12A5 (FIG. 14B), and GABRA1 (FIG. 14C), and expressed high levels of proliferative mesenchymal subtype markers such as EGFR (FIG. 15A) and AKT1 (FIG. 15B). Further, markers for two pathways were upregulated in the tumor area. In particular, there was an increase in expression of markers for the NF-kB signaling pathway p65 (RELA) (FIG. 16A) and p50 (NFKB1) (FIG. 16B) and the MAPK signaling pathway MAPK1 (FIG. 16C). Taken together, these data demonstrate that the sandwiching methods used herein enabled unbiased transfer of ligation products which translates to classification vie gene expression profiles of the tumor type.

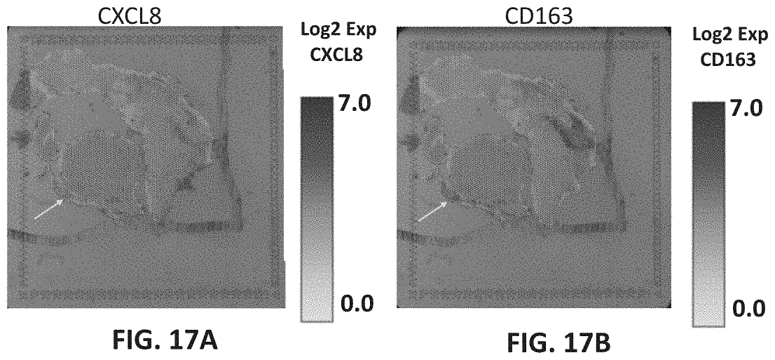

Next, metastatic markers were examined. As shown in FIG. 17A, CXCL8 was upregulated in cells with high metastatic potential. Spatial analysis identified a region in the bigger tumor that expressed high levels of CXCL8. The CXCL8 high cells also expressed high CD163 (FIG. 17B), a marker of macrophages with high metastatic potential. The data suggests that the sandwiching methods described herein can be used to transfer ligated probes to a spatial array in order to aid in identifying the spatial distribution of cells undergoing metastasis.

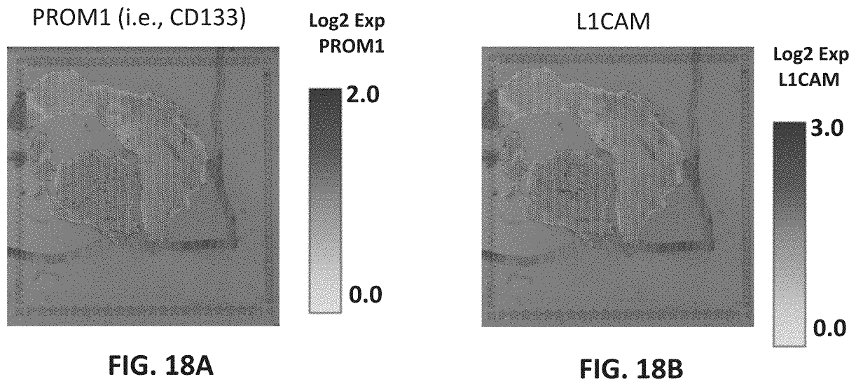

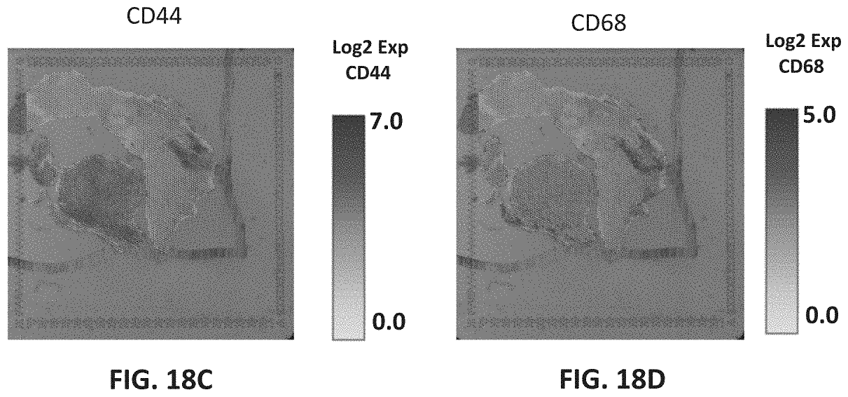

CD133 is a marker of Glioblastoma Stem Cells (GSC). Spatial data showed a rare population of cells in the tumor which expressed CD133 (i.e., PROM1; FIG. 18A), suggesting the sandwiching methods enabled the transfer of biomarkers to an arrayed slide where identification of glioblastoma stem cells was made possible. L1CAM is a marker of tumor cells with tumor initiating potential and important for maintaining glioblastoma stem cell population. The sandwiching methods also facilitated the identification of a small population of L1CAM+ cells within the larger tumor. FIG. 18B. These data suggests that the sandwiching methods described herein can be used to transfer ligated probes to a spatial array in order to aid in identifying rare cell populations within a large tumor area.

Next, CD44 expression in glioblastoma is correlated with radio resistance and poor survival. Both the tumor masses showed high CD44 expression (FIG. 18C) suggesting markers related to outcome of therapies and survival can be identified using the sandwiching methods.

Finally, CD68 is a marker of tumor associated macrophages. The sandwiching methods identified CD68+ cells in both tumor masses. FIG. 18D. Subset of cells in the larger mass that resembled smaller tumor cells based on clustering, expressed CD68 to similar levels to smaller tumor, suggesting macrophages influenced cell migration.

These results demonstrate that spatial methods can identify one or more genes that in a glioblastoma sample that can be actionable in determining a treatment regimen for a subject by a diagnostician. The data provide evidence that the methods used herein can be used to help identify various grades of tumor, follow its progression over time, identify subpopulations of cells in a tumor, and elucidate biomarkers and pathways associated with glioblastoma progression when the original sample is found on a standard slide.

What is claimed is:

1. A method of identifying a location of a disease proliferating region in a glioblastoma-derived sample, comprising:
(a) providing the glioblastoma-derived sample on a first substrate;
(b) contacting a first probe and a second probe with the glioblastoma-derived sample, wherein the first probe and the second probe each comprise one or more sequences that are substantially complementary to sequences of a nucleic acid indicative of a disease proliferating region in a glioblastoma, and wherein the second probe comprises a capture probe capture domain;
(c) hybridizing the first probe and the second probe to the nucleic acid indicative of a disease proliferating region in the glioblastoma;
(d) generating a ligation product by ligating the first probe and the second probe;
(e) releasing the ligated product from the nucleic acid indicative of a disease proliferating region in the glioblastoma;
(f) hybridizing the ligation product to a capture domain affixed to an array; and
(g) determining the sequences (i) all or a part of the ligation product hybridized to the capture domain, or a complement thereof, and (ii) a spatial barcode, or a complement thereof, and using the determined sequences of (i) and (ii) to identify the location of the disease proliferating region in the glioblastoma-derived sample.

2. The method of claim 1, wherein the nucleic acid indicative of the disease proliferating region in the glioblastoma-derived sample is an mRNA that codes for one or more of Ki67, CCNB1, and MYCC.

3. The method of claim 1, wherein the disease proliferating region is a region of metastasis.

4. The method of claim 1, wherein the glioblastoma-derived sample is a formalin-fixed paraffin-embedded (FFPE) glioblastoma sample, a PFA fixed glioblastoma sample, or an acetone fixed glioblastoma sample.

5. The method of claim 4, wherein the glioblastoma-derived sample is an FFPE sample.

6. The method of claim 1, wherein the first substrate comprises the array comprising a plurality of capture probes, wherein a capture probe of the plurality of capture probes comprises (i) the spatial barcode and (ii) the capture domain.

7. The method of claim 1, further comprising:
aligning the first substrate with a second substrate comprising the array, such that at least a portion of the glioblastoma-derived sample is aligned with at least a portion of the array, wherein the array comprises a plurality of capture probes, wherein a capture probe of the plurality of capture probes comprises (i) the spatial barcode and (ii) the capture domain; and
after the glioblastoma-derived sample is aligned with at least a portion of the array, performing the releasing step (e) and migrating the ligation product from the glioblastoma-derived sample to the array.

8. The method of claim 1, wherein the first probe and the second probe hybridize to a contiguous sequence on the nucleic acid.

9. The method of claim 1, wherein the first probe and the second probe hybridize to adjacent sequences on the nucleic acid.

10. The method of claim 1, wherein the ligating is via enzymatic ligation and is performed by an enzyme that is selected from a Chlorella virus DNA ligase, a single-stranded DNA ligase, or a T4 DNA ligase.

11. The method of claim 1, wherein the releasing step (e) comprises contacting the glioblastoma-derived sample with a reagent medium comprising a permeabilization agent and an agent for releasing the ligation product from the nucleic acid, thereby permeabilizing the glioblastoma-derived sample and releasing the ligation product from the nucleic acid.

12. The method of claim 11, wherein the agent for releasing the ligation product from the nucleic acid comprises an RNase selected from the group consisting of RNase A, RNase C, RNase H, or RNase I.

13. The method of claim 11, wherein the permeabilization agent comprises a protease selected from the group consisting of trypsin, pepsin, elastase, proteinase K, collagenase, or a combination thereof.

14. The method of claim 11, wherein the glioblastoma-derived sample is contacted with the reagent medium for about 1 to about 60 minutes.

15. The method of claim 1, wherein the determining comprises next generation sequencing.

16. The method of claim 1, wherein the capture domain comprises a poly(T) sequence, and wherein the capture domain comprises a sequence complementary to a portion of the first probe or a portion of the second probe.

17. The method of claim 1, wherein the capture probe further comprises one or more functional domains, a unique molecular identifier (UMI), a cleavage domain, or combinations thereof.

18. The method of claim 1, wherein the nucleic acid indicative of a disease proliferating region in a glioblastoma is mRNA.

19. The method of claim 1, further comprising staining and imaging the glioblastoma-derived sample using one or more of hematoxylin, eosin, immunohistochemistry, or immunofluorescence.

20. The method of claim 1, further comprising identifying a location of a protein in the glioblastoma-derived sample.

21. The method of claim 20, wherein identifying the location of the protein comprises:
contacting the glioblastoma-derived sample with a plurality of analyte capture agents, wherein an analyte capture agent of the plurality of analyte capture agents comprises an analyte binding moiety and an analyte binding moiety barcode domain, wherein the analyte binding moiety specifically binds to the protein, and wherein the analyte binding moiety barcode domain comprises a protein binding moiety barcode and a protein capture domain;
hybridizing the protein capture domain of the analyte binding moiety barcode domain to a second capture domain of a second capture probe, wherein the second capture probe further comprises a second spatial barcode; and
determining the sequences of (iii) all or a part of the analyte binding moiety barcode domain, or a complement thereof; and (iv) the second spatial barcode, or a complement thereof, and using the determined sequences of (iii) and (iv) to identify the location of the protein in the glioblastoma-derived sample.

22. The method of claim 21, wherein the protein is one or more protein biomarkers selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.

23. A method of identifying a location of a nucleic acid in a glioblastoma sample, the method comprising:
(a) providing the glioblastoma sample on a first substrate;
(b) contacting a first probe and a second probe with the glioblastoma sample, wherein the first probe and the second probe each comprise one or more sequences that are substantially complementary to sequences of the nucleic acid, and wherein the second probe comprises a capture probe capture domain;
(c) hybridizing the first probe and the second probe to the nucleic acid;
(d) generating a ligation product by ligating the first probe and the second probe;
(e) aligning the first substrate with a second substrate comprising an array, such that at least a portion of the glioblastoma sample is aligned with at least a portion of the array, wherein the array comprises a plurality of capture probes, wherein a capture probe of the plurality of capture probes comprises (i) a spatial barcode and (ii) the capture domain;
(f) releasing the ligated product from the nucleic acid;
(g) hybridizing the ligation product to the capture domain of the capture probe on the array; and
(h) determining (i) all or a part of the sequence of the ligation product hybridized to the capture domain, or a complement thereof, and (ii) a spatial barcode, or a complement thereof, and using the determined sequences of (i) and (ii) to identify the location of the nucleic acid in the glioblastoma sample.

24. The method of claim 23, wherein the glioblastoma sample is an archived fixed glioblastoma sample that has been stored on the first substrate for at least six months.

25. The method of claim 23, wherein the glioblastoma sample is an archived fixed glioblastoma sample that has been stored on the first substrate for at least one year.

26. The method of claim 24, wherein the archived fixed glioblastoma sample has been stored on the first substrate at room temperature.

27. The method of claim 24, wherein the archived fixed glioblastoma sample has been stored on the first substrate at a temperature above room temperature.

28. The method of claim 25, wherein the archived fixed glioblastoma sample has been stored on the first substrate at room temperature.

29. The method of claim 25, wherein the archived fixed glioblastoma sample has been stored on the first substrate above room temperature.

30. A composition comprising:
(a) a biological sample placed on a first substrate, wherein the biological sample comprises an analyte;
(b) a second substrate comprising an array comprising a plurality of capture probes affixed to the second substrate, wherein a capture probe of the plurality of capture probes comprises (i) a spatial barcode comprising a sequence that provides a location of the analyte and (ii) a capture domain, wherein the first substrate is aligned with the second substrate, such that at least a portion of the biological sample is aligned with at least a portion of the array; and
(c) a ligation product comprising a first probe and a second probe, wherein the first probe and the second probe each comprise a sequence that is substantially complementary to a sequence of the analyte, and wherein one of the first probe or the second probe comprises a capture probe capture domain that is hybridized to the capture domain of the capture probe on the second substrate,
wherein the analyte is one or more biomarkers selected from TAF11L1, Ki67, CCNB1, MYCC, MYCN, SYT1, SLC12A5, GABRA1, EGFR, AKT1, p65 (RELA), p50 (NFKB1), MAPK1, CXCL8, CD163, PROM1, L1CAM, CD44, CD68, or any combination thereof.
